SEQUENCE LISTING (165 sequences)

SEQ ID NO: 1 - protein, 124 aa, synthetic construct
QVQLVESGGGLVQAGGSLRLSCTGSARPFSNYGMGWFRQAPGKEREFVAAISGGGVYTDFADAVKGRFTISRDNAKNTVYLQMNSLKPEDTAVYYCARARTARVVSRRHEYDYWGQGTQVTVSS

SEQ ID NO: 2 - protein, 124 aa, synthetic construct
QVQLVESGGGLVQAGGSLRLSCTASGGTFVNYGMGWFRQAPGKEREFVAAISGGGVYRDFADSVKGRFTISRDNAKNTVYLQMNSLKPEDTALYYCARARTPRVVNRRHEYDYWGQGTQVTVSS

SEQ ID NO: 3 - protein, 124 aa, synthetic construct
QVQFVESGGGLVQAGGSLRLSCTASGGIFSNYGMGWFRQAPGKEREFVAAISGGGVYTDFADSVKGRFTISRDRAKNTVYLQMNSLKPEDTAVYYCARARTARLTSRRHDYDYWGQGTQVTVSS

SEQ ID NO: 4 - protein, 125 aa, synthetic construct
QSQLVESGGGLVQPGGSLRLSCAASGSTLDHYATAWFRRAPGKEREGISYISSGGGSTYYVDSVKGRFTISRDNAKRTVYLQMNSLKPEDTAVYFCASKRRFNTGWSEPSDYNVWGQGTQVTVTS

SEQ ID NO: 5 - protein, 115 aa, synthetic construct
AVQLVESGGGLVQPGGSLRLSCAASGSTISINVMGWYRQAPGKQRDLVAAISSVGSTYYADSVKGRFTISRDNAKNTVYLQMNSLKPEDTAVYYCNAKSLISDYWGQGTQVTVSS

SEQ ID NO: 6 - protein, 122 aa, synthetic construct
QVQVVESGGGLVQAGGSLRLSCAASGRAFSSYAMGWFRQAPGKQRELVAVITSGGSTNYADSVKGRFTISRDNAKNTVYLQMNSLKPEDTAVYYCAAGTGYGGGFLIGYDYWGQGTQVTVSS

SEQ ID NO: 7 - protein, 113 aa, synthetic construct
QVQLVESGGGLVQPGGSLRLSCAASGSRFMTSSMGWYRQTPGKQREWVATATSSDSTLYADAVKGRFTISRDNAKNTVYLQMNSLKPQDTAVYYCFTTPPTFWGHGTQVTVSS

SEQ ID NO: 8 - protein, 128 aa, synthetic construct
EVDLVESGGGLVQPGGSLRLSCGASGFTFSHYAMSWVRQAPGKALEWVSDISSGGGITKYADSVKGRFTISRDDAQNTLYLQMNSLKPEDTAVYYCAKASAIGYLAYWMRNPVEYDTRGQGTLVTVSS

SEQ ID NO: 9 - protein, 127 aa, synthetic construct
QKQLVESGGGLVQPGGSLRLSCAASGETLSNYNIGWFRQAPGKEREGVSCISQIGFTNYADSVKGRFTLFRASAENTVYLQMNSLKPEDTAVYYCAVTGGRWASCSFLPLRQDFESWGQGTLVTVSS

SEQ ID NO: 10 - protein, 115 aa, synthetic construct
AVQLVESGGGMVQPGGSLRLSCAASGFTFGAYDMTWVRQAPGKGAEWVSAINSGGDRTYYTDSVKGRFTISRDNAKNTLYLQMNSLKPADTAKYYCGTKIPQFLRGQGTQVTVSS

SEQ ID NO: 11 - protein, 8 aa, synthetic construct
ARPFSNYG

SEQ ID NO: 12 - protein, 8 aa, synthetic construct
GGTFVNYG

SEQ ID NO: 13 - protein, 8 aa, synthetic construct
GGIFSNYG

SEQ ID NO: 14 - protein, 8 aa, synthetic construct
GSTLDHYA

SEQ ID NO: 15 - protein, 8 aa, synthetic construct
GSTISINV

SEQ ID NO: 16 - protein, 8 aa, synthetic construct
GRAFSSYA

SEQ ID NO: 17 - protein, 8 aa, synthetic construct
GSRFMTSS

SEQ ID NO: 18 - protein, 8 aa, synthetic construct
GFTFSHYA

SEQ ID NO: 19 - protein, 8 aa, synthetic construct
GETLSNYN

SEQ ID NO: 20 - protein, 8 aa, synthetic construct
GFTFGAYD

SEQ ID NO: 21 - protein, 8 aa, synthetic construct
ISGGGVYT

SEQ ID NO: 22 - protein, 8 aa, synthetic construct
ISGGGVYR

SEQ ID NO: 23 - protein, 8 aa, synthetic construct
ISGGGVYT

SEQ ID NO: 24 - protein, 8 aa, synthetic construct
ISSGGGST

SEQ ID NO: 25 - protein, 7 aa, synthetic construct
ISSVGST

SEQ ID NO: 26 - protein, 7 aa, synthetic construct
ITSGGST

SEQ ID NO: 27 - protein, 7 aa, synthetic construct
ATSSDST

SEQ ID NO: 28 - protein, 8 aa, synthetic construct
ISSGGGIT

SEQ ID NO: 29 - protein, 7 aa, synthetic construct
ISQIGFT

SEQ ID NO: 30 - protein, 8 aa, synthetic construct
INSGGDRT

SEQ ID NO: 31 - protein, 17 aa, synthetic construct
ARARTARVVSRRHEYDY

SEQ ID NO: 32 - protein, 17 aa, synthetic construct
ARARTPRVVNRRHEYDY

SEQ ID NO: 33 - protein, 17 aa, synthetic construct
ARARTARLTSRRHDYDY

SEQ ID NO: 34 - protein, 18 aa, synthetic construct
ASKRRFNTGWSEPSDYNV

SEQ ID NO: 35 - protein, 9 aa, synthetic construct
NAKSLISDY

SEQ ID NO: 36 - protein, 16 aa, synthetic construct
AAGTGYGGGFLIGYDY

SEQ ID NO: 37 - protein, 7 aa, synthetic construct
FTTPPTF

SEQ ID NO: 38 - protein, 21 aa, synthetic construct
AKASAIGYLAYWMRNPVEYDT

SEQ ID NO: 39 - protein, 21 aa, synthetic construct
AVTGGRWASCSFLPLRQDFES

SEQ ID NO: 40 - protein, 8 aa, synthetic construct
GTKIPQFL

SEQ ID NO: 41 - DNA, 372 bp, synthetic construct
  source 1..372: mol_type = other DNA
caggtgcagctcgtggaatctgggggaggattggtgcaggccggggggtctctgagactctcctgtacaggctctgcacgccccttcagtaactatggcatgggctggttccgccaggctccagggaaggagcgtgagtttgttgcagctattagcggtggtggtgtctacacagacttcgcagacgccgtgaagggccgattcaccatctccagagacaatgccaagaacacggtgtatctgcaaatgaacagcctgaaacctgaggacacggccgtttattactgtgcacgcgcgcgtacagcgcgcgttgttagtagacgtcatgagtatgactactggggccaggggacccaggtcaccgtctcctca

SEQ ID NO: 42 - DNA, 372 bp, synthetic construct
  source 1..372: mol_type = other DNA
caggtgcagcttgtggagtctgggggaggattggtgcaggccggggggtctctgagactctcctgtacagcctctggaggcacctttgttaactatggcatgggctggttccgccaggctccagggaaggagcgtgagtttgttgcagctattagcggtggtggtgtctaccgagacttcgcagactccgtgaagggccgattcaccatctccagagacaatgccaagaacacggtgtatctgcaaatgaacagcctgaaacctgaggacacggccctttattactgtgcacgcgcgcgtacacctcgtgttgttaatagacgtcatgagtatgactactggggccaggggacccaggtcaccgtctcctca

SEQ ID NO: 43 - DNA, 372 bp, synthetic construct
  source 1..372: mol_type = other DNA
caggtgcagttcgtggaatctgggggaggattggtgcaggccggggggtctctgagactctcctgtacagcctctggaggcatcttcagtaactatggcatgggctggttccgccaggctccagggaaggagcgtgagtttgttgcagctattagcggtggtggtgtctacacagacttcgcagactccgtgaagggccgattcaccatctccagagaccgtgccaagaacacggtgtatctgcaaatgaacagcctgaaacctgaggacacggccgtttattactgtgcacgcgcgcgtacagcacggcttactagtagacgtcatgactatgactactggggccaggggacccaggtcaccgtctcctca

SEQ ID NO: 44 - DNA, 375 bp, synthetic construct
  source 1..375: mol_type = other DNA
cagtcgcagctcgtggaatctgggggaggcttggtgcagcctggggggtctctgagactctcctgtgcagcctctggatccactttggatcattatgccactgcctggttccgccgggccccagggaaggagcgtgaggggatctcatatattagtagtggtggtggtagcacgtactatgtagactccgtgaagggccgattcaccatctccagagacaacgccaagaggacggtgtatctgcaaatgaacagcctgaaacctgaggacacagccgtttatttctgtgcgtccaagagaaggttcaatactggctggtccgagccttcagattataacgtctggggccaggggacccaggtcaccgtcacctca

SEQ ID NO: 45 - DNA, 345 bp, synthetic construct
  source 1..345: mol_type = other DNA
gcggtacaactggtggagtctgggggaggcttggtgcagcctggggggtctctgagactctcctgtgcagcctccggaagtacaatcagtatcaatgtcatgggctggtaccgccaggctccagggaagcagcgcgacttggtcgcagctattagtagtgttggtagcacatactatgcagactccgtgaagggccgattcaccatctccagagacaacgccaagaacacggtgtatctgcaaatgaacagcctgaaacctgaggacacggccgtctattactgtaatgccaagtcactcataagtgactactggggccagggtacccaggtcaccgtctcctca

SEQ ID NO: 46 - DNA, 366 bp, synthetic construct
  source 1..366: mol_type = other DNA
caggtgcaggtcgtggaatctgggggaggattggtgcaggctgggggctctctgagactctcctgtgcagcctctggacgcgccttcagtagctatgccatgggctggttccgccaggctccagggaagcagcgcgagttggtcgcagttattactagtggtggtagcacaaactatgcagactccgtgaagggccgattcaccatctccagagacaacgccaagaacacggtgtatctgcaaatgaacagcctgaaacctgaggacacggccgtttattactgtgcagcagggaccggatacgggggggggtttttaattgggtatgactactggggccagggtacccaggtcaccgtctcctca

SEQ ID NO: 47 - DNA, 339 bp, synthetic construct
  source 1..339: mol_type = other DNA
caggtgcagctggtggagtctgggggaggcttggtgcagcctggggggtccctgagactctcctgtgcagcctctggaagccgcttcatgacgagttccatgggctggtaccgccagactccaggaaaacagcgcgagtgggtcgcgactgctactagtagtgatagtacactttatgcagacgccgtgaagggccgattcaccatctccagagacaacgccaagaacacggtgtatttgcaaatgaacagcctgaaaccgcaggacacggccgtctattactgtttcacaacaccacccaccttctggggccacgggacccaggtcaccgtctcctca

SEQ ID NO: 48 - DNA, 384 bp, synthetic construct
  source 1..384: mol_type = other DNA
gaggtagatcttgtggagtctgggggaggcttggtgcagcctggggggtctctgagactctcctgtggagcctctggattcaccttcagtcactatgccatgagctgggtccgccaggctccaggaaaggcactcgagtgggtctcagatattagtagtggtggtggtattacaaagtatgcagactccgtgaagggccgattcaccatctccagagacgacgcccagaacacgctgtatctgcaaatgaatagcctgaaacctgaggacacggccgtctattactgtgcgaaagccagtgccataggatatctcgcctattggatgaggaaccccgtagagtatgacacccggggacaggggaccctggtcactgtctcctca

SEQ ID NO: 49 - DNA, 381 bp, synthetic construct
  source 1..381: mol_type = other DNA
cagaagcagctcgtggagtctgggggaggcttggtgcagcctggggggtctctgagactctcctgtgcagcctctggagagactttgtccaattataacataggctggttccgccaggccccaggcaaggagcgtgagggggtctcatgcattagtcagattggtttcaccaactatgcagactccgtgaagggccgattcaccctctttcgagcgagcgccgagaacacggtgtatctgcaaatgaacagcctgaaacctgaggacacagccgtttattactgtgcagtgacgggcggaagatgggctagctgcagttttctgccacttcgccaggactttgagtcctggggccaggggaccctggtcactgtctcctca

SEQ ID NO: 50 - DNA, 345 bp, synthetic construct
  source 1..345: mol_type = other DNA
gcggtacaactggtggagtctgggggaggcatggtgcagccgggggggtctctgagactctcctgtgcagcctctggattcaccttcggagcctatgacatgacctgggtccgccaggctccaggaaagggagccgagtgggtctcagctattaatagtggtggtgaccgcacatactatacagactccgtgaagggccgattcaccatctccagagacaacgccaagaacacgctgtatctgcaaatgaacagcctgaaacctgcggacacggccaagtattactgtggaaccaaaatcccgcagttcctcaggggccaggggacccaggtcaccgtctcctca

SEQ ID NO: 51 - protein, 5 aa, synthetic construct
NYGMG

SEQ ID NO: 52 - protein, 5 aa, synthetic construct
NYGMG

SEQ ID NO: 53 - protein, 5 aa, synthetic construct
NYGMG

SEQ ID NO: 54 - protein, 5 aa, synthetic construct
HYATA

SEQ ID NO: 55 - protein, 16 aa, synthetic construct
AISSVGSTYYADSVKG

SEQ ID NO: 56 - protein, 5 aa, synthetic construct
SYAMG

SEQ ID NO: 57 - protein, 5 aa, synthetic construct
TSSMG

SEQ ID NO: 58 - protein, 5 aa, synthetic construct
HYAMS

SEQ ID NO: 59 - protein, 5 aa, synthetic construct
NYNIG

SEQ ID NO: 60 - protein, 5 aa, synthetic construct
AYDMT

SEQ ID NO: 61 - protein, 17 aa, synthetic construct
AISGGGVYTDFADAVKG

SEQ ID NO: 62 - protein, 17 aa, synthetic construct
AISGGGVYRDFADSVKG

SEQ ID NO: 63 - protein, 17 aa, synthetic construct
AISGGGVYTDFADSVKG

SEQ ID NO: 64 - protein, 17 aa, synthetic construct
YISSGGGSTYYVDSVKG

SEQ ID NO: 65 - protein, 16 aa, synthetic construct
AISSVGSTYYADSVKG

SEQ ID NO: 66 - protein, 16 aa, synthetic construct
VITSGGSTNYADSVKG

SEQ ID NO: 67 - protein, 16 aa, synthetic construct
TATSSDSTLYADAVKG

SEQ ID NO: 68 - protein, 17 aa, synthetic construct
DISSGGGITKYADSVKG

SEQ ID NO: 69 - protein, 16 aa, synthetic construct
CISQIGFTNYADSVKG

SEQ ID NO: 70 - protein, 17 aa, synthetic construct
AINSGGDRTYYTDSVKG

SEQ ID NO: 71 - protein, 15 aa, synthetic construct
ARTARVVSRRHEYDY

SEQ ID NO: 72 - protein, 15 aa, synthetic construct
ARTPRVVNRRHEYDY

SEQ ID NO: 73 - protein, 15 aa, synthetic construct
ARTARLTSRRHDYDY

SEQ ID NO: 74 - protein, 16 aa, synthetic construct
KRRFNTGWSEPSDYNV

SEQ ID NO: 75 - protein, 7 aa, synthetic construct
KSLISDY

SEQ ID NO: 76 - protein, 14 aa, synthetic construct
GTGYGGGFLIGYDY

SEQ ID NO: 77 - protein, 5 aa, synthetic construct
TPPTF

SEQ ID NO: 78 - protein, 19 aa, synthetic construct
ASAIGYLAYWMRNPVEYDT

SEQ ID NO: 79 - protein, 19 aa, synthetic construct
TGGRWASCSFLPLRQDFES

SEQ ID NO: 80 - protein, 6 aa, synthetic construct
KIPQFL

SEQ ID NO: 81 - protein, 407 aa, synthetic construct
SQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQ

SEQ ID NO: 82 - protein, 124 aa, synthetic construct
EVQLVESGGGLVQPGGSLRLSCAASARPFSNYGMSWVRQAPGKGLEWVSAISGGGVYTYYADSVKGRFTISRDNSKNTLYLQMNSLRAEDTAVYYCARARTARVVSRRHEYDYWGQGTQVTVSS

SEQ ID NO: 83 - protein, 124 aa, synthetic construct
EVQLVESGGGLVQPGGSLRLSCAASARPFSNYGMGWFRQAPGKEREFVAAISGGGVYTYYADSVKGRFTISRDNSKNTLYLQMNSLRAEDTAVYYCARARTARVVSRRHEYDYWGQGTQVTVSS

SEQ ID NO: 84 - protein, 124 aa, synthetic construct
EVQLVESGGGLVQPGGSLRLSCTGSARPFSNYGMGWFRQAPGKEREFVAAISGGGVYTDFADSVKGRFTISRDNSKNTLYLQMNSLRAEDTAVYYCARARTARVVSRRHEYDYWGQGTQVTVSS

SEQ ID NO: 85 - protein, 124 aa, synthetic construct
EVQLVESGGGLVQPGGSLRLSCAASARPFSNYGMGWFRQAPGKEREFVAAISGGGVYTYYADSVKGRFTISRDNSRNTLYLQMNSLRAEDTAVYYCARARTARVVSRRHEYDYWGQGTQVTVSS

SEQ ID NO: 86 - protein, 124 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSS

SEQ ID NO: 87 - protein, 124 aa, synthetic construct
EVQLVESGGGLVQPGGSLRLSCAASGGTFVNYGMSWVRQAPGKGLEWVSAISGGGVYRYYADSVKGRFTISRDNSKNTLYLQMNSLRAEDTAVYYCARARTPRVVNRRHEYDYWGQGTQVTVSS

SEQ ID NO: 88 - protein, 124 aa, synthetic construct
EVQLVESGGGLVQPGGSLRLSCAASGGTFVNYGMGWFRQAPGKEREFVAAISGGGVYRYYADSVKGRFTISRDNSKNTLYLQMNSLRAEDTAVYYCARARTPRVVNRRHEYDYWGQGTQVTVSS

SEQ ID NO: 89 - protein, 124 aa, synthetic construct
EVQLVESGGGLVQPGGSLRLSCTASGGTFVNYGMGWFRQAPGKEREFVAAISGGGVYRDFADSVKGRFTISRDNSKNTLYLQMNSLRAEDTAVYYCARARTPRVVNRRHEYDYWGQGTQVTVSS

SEQ ID NO: 90 - protein, 124 aa, synthetic construct
EVQLVESGGGLVQPGGSLRLSCAASGGTFVNYGMGWFRQAPGKEREFVAAISGGGVYRYYADSVKGRFTISRDNSRNTLYLQMNSLRAEDTAVYYCARARTPRVVNRRHEYDYWGQGTQVTVSS

SEQ ID NO: 91 - protein, 124 aa, synthetic construct
EVQLVESGGGLVQPGGSLRLSCAASGGIFSNYGMSWVRQAPGKGLEWVSAISGGGVYTYYADSVKGRFTISRDNSKNTLYLQMNSLRAEDTAVYYCARARTARLTSRRHDYDYWGQGTQVTVSS

SEQ ID NO: 92 - protein, 124 aa, synthetic construct
EVQLVESGGGLVQPGGSLRLSCAASGGIFSNYGMGWFRQAPGKEREFVAAISGGGVYTYYADSVKGRFTISRDNSKNTLYLQMNSLRAEDTAVYYCARARTARLTSRRHDYDYWGQGTQVTVSS

SEQ ID NO: 93 - protein, 124 aa, synthetic construct
EVQLVESGGGLVQPGGSLRLSCTASGGIFSNYGMGWFRQAPGKEREFVAAISGGGVYTDFADSVKGRFTISRDNSKNTLYLQMNSLRAEDTAVYYCARARTARLTSRRHDYDYWGQGTQVTVSS

SEQ ID NO: 94 - protein, 124 aa, synthetic construct
EVQLVESGGGLVQPGGSLRLSCAASGGIFSNYGMGWFRQAPGKEREFVAAISGGGVYTYYADSVKGRFTISRDNSRNTLYLQMNSLRAEDTAVYYCARARTARLTSRRHDYDYWGQGTQVTVSS

SEQ ID NO: 95 - protein, 125 aa, synthetic construct
QSQLVESGGGLVQPGGSLRLSCAASGSTLDHYAMSWVRQAPGKGLEWVSAISSGGGSTYYADSVKGRFTISRDNSKNTLYLQMNSLRAEDTAVYYCASKRRFNTGWSEPSDYNVWGQGTQVTVTS

SEQ ID NO: 96 - protein, 125 aa, synthetic construct
QSQLVESGGGLVQPGGSLRLSCAASGSTLDHYATAWFRRAPGKEREGISYISSGGGSTYYADSVKGRFTISRDNSKNTLYLQMNSLRAEDTAVYYCASKRRFNTGWSEPSDYNVWGQGTQVTVTS

SEQ ID NO: 97 - protein, 125 aa, synthetic construct
QSQLVESGGGLVQPGGSLRLSCAASGSTLDHYATAWFRRAPGKEREGISYISSGGGSTYYVDSVKGRFTISRDNSKNTLYLQMNSLRAEDTAVYFCASKRRFNTGWSEPSDYNVWGQGTQVTVTS

SEQ ID NO: 98 - protein, 125 aa, synthetic construct
QSQLVESGGGLVQPGGSLRLSCAASGSTLDHYATAWFRRAPGKEREGISYISSGGGSTYYADSVAGRFTISRDNSRNTLYLQMNSLRAEDTAVYYCASKRRFNTGWSEPSDYNVWGQGTQVTVTS

SEQ ID NO: 99 - protein, 115 aa, synthetic construct
AVQLVESGGGLVQPGGSLRLSCAASGSTISINVMSWVRQAPGKGLEWVSVISSVGSTYYADSVKGRFTISRDNSKNTLYLQMNSLRAEDTAVYYCNAKSLISDYWGQGTQVTVSS

SEQ ID NO: 100 - protein, 115 aa, synthetic construct
AVQLVESGGGLVQPGGSLRLSCAASGSTISINVMGWYRQAPGKQRDLVAAISSVGSTYYADSVKGRFTISRDNSKNTLYLQMNSLRAEDTAVYYCNAKSLISDYWGQGTQVTVSS

SEQ ID NO: 101 - protein, 115 aa, synthetic construct
AVQLVESGGGLVQPGGSLRLSCAASGSTISINVMGWYRQAPGKQRDLVAAISSVGSTYYADSVAGRFTISRDNSRNTLYLQMNSLRAEDTAVYYCNAKSLISDYWGQGTQVTVSS

SEQ ID NO: 102 - protein, 122 aa, synthetic construct
EVQLVESGGGLVQPGGSLRLSCAASGRAFSSYAMSWVRQAPGKGLEWVSAITSGGSTYYADSVKGRFTISRDNSKNTLYLQMNSLRAEDTAVYYCAAGTGYGGGFLIGYDYWGQGTQVTVSS

SEQ ID NO: 103 - protein, 122 aa, synthetic construct
EVQLVESGGGLVQPGGSLRLSCAASGRAFSSYAMGWFRQAPGKQRELVAVITSGGSTYYADSVKGRFTISRDNSKNTLYLQMNSLRAEDTAVYYCAAGTGYGGGFLIGYDYWGQGTQVTVSS

SEQ ID NO: 104 - protein, 122 aa, synthetic construct
EVQLVESGGGLVQPGGSLRLSCAASGRAFSSYAMGWFRQAPGKQRELVAVITSGGSTNYADSVKGRFTISRDNSKNTLYLQMNSLRAEDTAVYYCAAGTGYGGGFLIGYDYWGQGTQVTVSS

SEQ ID NO: 105 - protein, 122 aa, synthetic construct
EVQLVESGGGLVQPGGSLRLSCAASGRAFSSYAMGWFRQAPGKQRELVAVITSGGSTYYADSVKGRFTISRDNSRNTLYLQMNSLRAEDTAVYYCAAGTGYGGGFLIGYDYWGQGTQVTVSS

SEQ ID NO: 106 - protein, 113 aa, synthetic construct
EVQLVESGGGLVQPGGSLRLSCAASGSRFMTSSMNWVRQAPGKGLEWVSTATSSDSTLYADSVKGRFTISRDNAKNSLYLQMNSLRAEDTAVYYCFTTPPTFWGHGTQVTVSS

SEQ ID NO: 107 - protein, 113 aa, synthetic construct
EVQLVESGGGLVQPGGSLRLSCAASGSRFMTSSMGWYRQTPGKQREWVATATSSDSTLYADSVKGRFTISRDNAKNSLYLQMNSLRAEDTAVYYCFTTPPTFWGHGTQVTVSS

SEQ ID NO: 108 - protein, 113 aa, synthetic construct
EVQLVESGGGLVQPGGSLRLSCAASGSRFMTSSMGWYRQTPGKQREWVATATSSDSTLYADSVKGRFTISRDNARNSLYLQMNSLRAEDTAVYYCFTTPPTFWGHGTQVTVSS

SEQ ID NO: 109 - protein, 113 aa, synthetic construct
EVQLVESGGGLVQPGGSLRLSCAASGSRFMTSSMGWYRQTPGNQREWVATATSSDSTLYADSVKGRFTISRDNAKNSLYLQMNSLRAEDTAVYYCFTTPPTFWGHGTQVTVSS

SEQ ID NO: 110 - protein, 113 aa, synthetic construct
EVQLVESGGGLVQPGGSLRLSCAASGSRFMTSSMGWYRQTPGNGREWVATATSSDSTLYADSVKGRFTISRDNAKNSLYLQMNSLRAEDTAVYYCFTTPPTFWGHGTQVTVSS

SEQ ID NO: 111 - protein, 128 aa, synthetic construct
EVQLVESGGGLVQPGGSLRLSCAASGFTFSHYAMSWVRQAPGKGLEWVSAISSGGGITYYADSVKGRFTISRDNSKNTLYLQMNSLRAEDTAVYYCAKASAIGYLAYWMRNPVEYDTRGQGTLVTVSS

SEQ ID NO: 112 - protein, 128 aa, synthetic construct
EVQLVESGGGLVQPGGSLRLSCAASGFTFSHYAMSWVRQAPGKALEWVSDISSGGGITYYADSVKGRFTISRDNSKNTLYLQMNSLRAEDTAVYYCAKASAIGYLAYWMRNPVEYDTRGQGTLVTVSS

SEQ ID NO: 113 - protein, 128 aa, synthetic construct
EVQLVESGGGLVQPGGSLRLSCGASGFTFSHYAMSWVRQAPGKALEWVSDISSGGGITKYADSVKGRFTISRDNSKNTLYLQMNSLRAEDTAVYYCAKASAIGYLAYWMRNPVEYDTRGQGTLVTVSS

SEQ ID NO: 114 - protein, 128 aa, synthetic construct
QVQLVESGGGLVQPGGSLRLSCAASGFTFSHYAMSWVRQAPGKALEWVSDISSGGGITYYADSVAGRFTISRDNSRNTLYLQMNSLRAEDTAVYYCAKASAIGYLAYWMRNPVEYDTRGQGTLVTVSS

SEQ ID NO: 115 - protein, 127 aa, synthetic construct
QVQLVESGGGLVKPGGSLRLSCAASGETLSNYNMSWIRQAPGKGLEWVSYISQIGFTNYADSVKGRFTISRDNAKNSLYLQMNSLRAEDTAVYYCAVTGGRWASCSFLPLRQDFESWGQGTLVTVSS

SEQ ID NO: 116 - protein, 127 aa, synthetic construct
QVQLVESGGGLVKPGGSLRLSCAASGETLSNYNIGWFRQAPGKEREGVSCISQIGFTNYADSVKGRFTISRDNAKNSLYLQMNSLRAEDTAVYYCAVTGGRWASCSFLPLRQDFESWGQGTLVTVSS

SEQ ID NO: 117 - protein, 127 aa, synthetic construct
QVQLVESGGGLVQPGGSLRLSCAASGETLSNYNIGWFRQAPGKEREGVSCISQIGFTNYADSVKGRFTISRDNARNSLYLQMNSLRAEDTAVYYCAVTGGRWASCSFLPLRQDFESWGQGTLVTVSS

SEQ ID NO: 118 - protein, 117 aa, synthetic construct
QPQVVESGGGLVQPGRSLTLSCAAYGTISKPYAMGWYRQAPEKQREWVATIFSDHSTNYAGAVKGRFTISRDNAKNTVYLQMNSLKPEDTAVYYCLPLGATFAGVAWGQGTQVTVSS

SEQ ID NO: 119 - protein, 118 aa, synthetic construct
EVDLVESGGGLVQPGGSLRLSCAASQSIFSINVISWYRQAPGKQRELVAAIHSGGSTNYADSVKGRFTISRDGTKNTVYLQMNSLKPEDTAVYYCHADTDFDRTFGSWGQGTQVTVSS

SEQ ID NO: 120 - protein, 117 aa, synthetic construct
QVQLVESGGGLVQPGGSLRLSCAASGFTFGTYDMVWVRQAPGKGAEWVSGINSGGGRTYYADSVKGRFTISRDNAKNTLYLQMNSLKSEDTAVYYCGTGDGRSRTYTGQGTQVTVSS

SEQ ID NO: 121 - protein, 126 aa, synthetic construct
EVQLVESGGGLVQAGDSLRLSCAASGGTFSNYAMGWFRQAPGKDREFVSIVSQIGGVTRYADSVKGRFTISRDNAKNMLYLQMNSLKPEDTAVYYCAAVRTNSRFYSDFEDSIVSWGQGTQVTVSS

SEQ ID NO: 122 - protein, 112 aa, synthetic construct
EVQLVESGGGLVQPGGSLRLSCAASGSRFMTNAMGWWRQAPGKQREWVATATSNSTLYAEAVKGRFTISRDNAKNTVYLQMNSLKPQDTGVYYCFTTPPTAWGHGTQVTVSS

SEQ ID NO: 123 - protein, 116 aa, synthetic construct
QVQLVESGGGVVQPGGSLRLSCAASGFTFGSYDMTWVRQAPGKGAEWVAAIDRGGTSTYYADSVKGRFTISRDNAKNTLYLQMNSLKPEDTAVYYCGTREDGRATTGQGTQVTVSS

SEQ ID NO: 124 - protein, 10 aa, synthetic construct
GTISKPYAMG

SEQ ID NO: 125 - protein, 10 aa, synthetic construct
QSIFSINVIS

SEQ ID NO: 126 - protein, 10 aa, synthetic construct
GFTFGTYDMV

SEQ ID NO: 127 - protein, 10 aa, synthetic construct
GGTFSNYAMG

SEQ ID NO: 128 - protein, 10 aa, synthetic construct
GSRFMTNAMG

SEQ ID NO: 129 - protein, 10 aa, synthetic construct
GFTFGSYDMT

SEQ ID NO: 130 - protein, 16 aa, synthetic construct
TIFSDHSTNYAGAVKG

SEQ ID NO: 131 - protein, 16 aa, synthetic construct
AIHSGGSTNYADSVKG

SEQ ID NO: 132 - protein, 17 aa, synthetic construct
GINSGGGRTYYADSVKG

SEQ ID NO: 133 - protein, 17 aa, synthetic construct
IVSQIGGVTRYADSVKG

SEQ ID NO: 134 - protein, 15 aa, synthetic construct
TATSNSTLYAEAVKG

SEQ ID NO: 135 - protein, 17 aa, synthetic construct
AIDRGGTSTYYADSVKG

SEQ ID NO: 136 - protein, 9 aa, synthetic construct
LGATFAGVA

SEQ ID NO: 137 - protein, 10 aa, synthetic construct
DTDFDRTFGS

SEQ ID NO: 138 - protein, 8 aa, synthetic construct
GDGRSRTY

SEQ ID NO: 139 - protein, 17 aa, synthetic construct
VRTNSRFYSDFEDSIVS

SEQ ID NO: 140 - protein, 5 aa, synthetic construct
TPPTA

SEQ ID NO: 141 - protein, 7 aa, synthetic construct
REDGRAT

SEQ ID NO: 142 - protein, 5 aa, synthetic construct
PYAMG

SEQ ID NO: 143 - protein, 5 aa, synthetic construct
INVIS

SEQ ID NO: 144 - protein, 5 aa, synthetic construct
TYDMV

SEQ ID NO: 145 - protein, 5 aa, synthetic construct
NYAMG

SEQ ID NO: 146 - protein, 5 aa, synthetic construct
TNAMG

SEQ ID NO: 147 - protein, 5 aa, synthetic construct
SYDMT

SEQ ID NO: 148 - protein, 16 aa, synthetic construct
TIFSDHSTNYAGAVKG

SEQ ID NO: 149 - protein, 16 aa, synthetic construct
AIHSGGSTNYADSVKG

SEQ ID NO: 150 - protein, 17 aa, synthetic construct
GINSGGGRTYYADSVKG

SEQ ID NO: 151 - protein, 17 aa, synthetic construct
IVSQIGGVTRYADSVKG

SEQ ID NO: 152 - protein, 15 aa, synthetic construct
TATSNSTLYAEAVKG

SEQ ID NO: 153 - protein, 17 aa, synthetic construct
AIDRGGTSTYYADSVKG

SEQ ID NO: 154 - protein, 9 aa, synthetic construct
LGATFAGVA

SEQ ID NO: 155 - protein, 10 aa, synthetic construct
DTDFDRTFGS

SEQ ID NO: 156 - protein, 8 aa, synthetic construct
GDGRSRTY

SEQ ID NO: 157 - protein, 17 aa, synthetic construct
VRTNSRFYSDFEDSIVS

SEQ ID NO: 158 - protein, 5 aa, synthetic construct
TPPTA

SEQ ID NO: 159 - protein, 7 aa, synthetic construct
REDGRAT

SEQ ID NO: 160 - DNA, 351 bp, synthetic construct
  source 1..351: mol_type = other DNA
cagccgcaggtcgtggagtctgggggaggcttggtgcagcctgggaggtctctgacactctcctgtgcagcctatggaaccatctcgaagccctatgccatgggctggtaccgccaggctccagagaagcagcgcgagtgggtcgcaactatttttagtgaccatagtacaaactatgcaggcgccgtgaagggccgattcaccatttccagagacaatgccaagaacacggtgtatctgcaaatgaacagcctgaaacctgaagacacggccgtctattattgtctcccattgggagcgacttttgccggtgtggcctggggccaggggacccaggtcaccgtctcctca

SEQ ID NO: 161 - DNA, 354 bp, synthetic construct
  source 1..354: mol_type = other DNA
gaggtagatctggtggagtctgggggaggcttggtgcagcctggggggtctctgagactctcctgtgcagcctctcaaagcatcttcagtatcaatgtcatatcctggtaccgccaggctccagggaagcagcgcgagttggtcgcagctattcatagtggtggtagcacaaactatgcagactccgtgaagggccgattcaccatctccagagacggcaccaagaatacggtgtatctgcaaatgaacagcctgaaacctgaggacacggccgtctattactgtcatgcagacaccgactttgaccggacctttggttcctggggccaggggacccaggtcaccgtctcctcg

SEQ ID NO: 162 - protein, 351 aa, synthetic construct
CAGGTGCAGCTTGTGGAATCTGGGGGAGGCTTGGTGCAGCCTGGGGGTTCTCTGAGACTCTCCTGTGCAGCCTCTGGATTCACCTTCGGAACCTATGACATGGTCTGGGTCCGCCAGGCTCCAGGAAAGGGGGCCGAGTGGGTCTCAGGTATTAATAGTGGTGGTGGTCGGACATACTATGCAGACTCCGTGAAGGGCCGCTTCACCATCTCCAGAGACAACGCCAAGAACACACTGTATCTGCAAATGAACAGCCTGAAATCTGAGGACACGGCCGTGTATTATTGTGGAACAGGCGACGGACGGTCCAGAACGTATACGGGCCAGGGGACCCAGGTCACCGTCTCCTCA

SEQ ID NO: 163 - protein, 378 aa, synthetic construct
GAGGTACAACTCGTGGAGTCTGGGGGAGGATTGGTGCAGGCTGGAGACTCTCTGAGACTCTCCTGTGCAGCCTCTGGTGGCACCTTCAGTAACTACGCCATGGGCTGGTTCCGCCAGGCTCCAGGGAAGGACCGTGAGTTTGTCTCAATTGTTAGCCAGATTGGTGGTGTCACACGCTATGCAGACTCCGTGAAGGGCCGATTCACCATCTCCAGAGACAACGCCAAGAATATGTTGTATCTGCAAATGAACAGCCTGAAACCTGAGGACACAGCCGTCTATTACTGTGCGGCAGTAAGGACTAATAGCAGGTTCTATAGCGACTTTGAAGATTCGATAGTTTCCTGGGGCCAGGGGACCCAGGTCACCGTCTCATCG

SEQ ID NO: 164 - protein, 336 aa, synthetic construct
GAGGTGCAGCTTGTGGAATCTGGGGGAGGCTTGGTGCAGCCTGGGGGGTCTCTGAGACTCTCCTGTGCAGCCTCTGGAAGTCGCTTCATGACGAATGCCATGGGCTGGTGGCGCCAGGCTCCAGGAAAGCAGCGCGAGTGGGTCGCAACAGCTACTAGTAATAGTACGCTTTATGCAGAAGCCGTGAAGGGCCGATTCACCATCTCCAGAGACAACGCCAAGAACACGGTGTATTTGCAAATGAACAGCCTGAAACCACAGGACACGGGCGTCTATTACTGTTTCACAACACCACCCACCGCCTGGGGCCACGGGACCCAGGTCACCGTCTCCTCA

SEQ ID NO: 165 - protein, 348 aa, synthetic construct
CAGGTGCAGCTTGTGGAATCTGGGGGAGGAGTGGTGCAGCCTGGGGGTTCTCTGAGACTCTCCTGTGCAGCCTCTGGATTCACCTTCGGAAGCTACGACATGACCTGGGTCCGCCAGGCTCCAGGAAAGGGGGCCGAGTGGGTCGCAGCTATAGATAGAGGTGGTACGAGCACATACTATGCAGACTCCGTGAAGGGCCGATTCACCATCTCCAGAGACAACGCCAAGAACACGCTGTATCTGCAAATGAACAGCCTGAAACCTGAGGACACGGCCGTGTATTACTGTGGAACTAGGGAGGACGGAAGAGCGACTACGGGCCAGGGGACCCAGGTCACCGTCTCCTCA

(19) United States
(12) Patent Application Publication
(10) Pub. No.: US 2026/0193377 A1
(43) Pub. Date: Jul. 9, 2026

(54) ANTIBODY SPECIFICALLY BINDING TO GCC

(71) Applicant: Full-life Technologies HK Limited, Hong Kong (CN)

(72) Inventors: Chung Lim WONG, Shanghai (CN); Ravi V. J. CHARI, Needham, MA (US); Chieh-Ju LEE, Douliou, Yunlin City (TW); Yihu XIE, Shanghai (CN)

(21) Appl. No.: 19/131,901

(22) PCT Filed: Nov. 23, 2023
(86) PCT No.: PCT/CN2023/133624

§ 371 Date: May 21, 2025

(30) Foreign Application Priority Data
Nov. 23, 2022 (WO) PCT/CN2022/133632

(51) Int. Cl. C07K 16/40

(52) U.S. Cl. CPC C07K 16/40; C07K 2317/24; C07K 2317/31; C07K 2317/52; C07K 2317/565; C07K 2317/569; C07K 2317/77

(57) ABSTRACT

Provided herein is an antibody specifically binding to guanylyl cyclase C (GCC), and a conjugate comprising the antibody.

### RELATED APPLICATIONS

The present application claims priority of International Application No. PCT/CN2022/133632, filed on Nov. 23, 2022, the disclosure of which is hereby incorporated by reference in its entirety.

### FIELD OF THE APPLICATION

The present application relates to an antibody specifically binding to Guanylate cyclase 2C (GCC). Also provided are methods of making and uses of the antibody.

### BACKGROUND OF THE APPLICATION

Guanylate cyclase 2C, also known as guanylyl cyclase C (GCC), intestinal guanylate cyclase, guanylate cyclase-C receptor, or the heat-stable enterotoxin receptor (hSTAR) is an enzyme that in human is encoded by the GUCY2C gene. It's expressed at the mucosal cells, e.g., at the apical brush border membranes, lining the small intestine, large intestine and rectum, which can regulate intestinal homeostasis and has emerged as a tumor suppressor. Further, it is universally expressed in advanced metastatic colorectal tumors, as well as other cancer types that arise through intestinal metaplasia, thereby satisfying many characteristics of a compelling target for treating malignancies.

### SUMMARY

In one aspect, provided herein is an antibody specifically binding to Guanylate cyclase 2C (GCC), comprising CDR1, CDR2 and CDR3 of a single domain antibody of anyone selected from the group consisting of SEQ ID NO. 1-10 and 118-123. In some embodiments, the CDR1, CDR2 and CDR3 are according to IMGT numbering. In some embodiments, the CDR1, CDR2 and CDR3 are according to Kabat numbering.

In another aspect, provided herein is an antibody specifically binding to Guanylate cyclase 2C (GCC) comprising: (1) a CDR1, which comprises an amino acid sequence selected from the group consisting of: (i) SEQ ID NO. 11-20 and 124-129, and (ii) an amino acid sequence with one or more amino acid alterations as compared to SEQ ID NO. 11-20 and 124-129; (2) a CDR2, which comprises an amino acid sequence selected from the group consisting of: (i) SEQ ID NO. 21-30 and 130-135, and (ii) an amino acid sequence with one or more amino acid alterations as compared to SEQ ID NO. 21-30 and 130-135; and (3) a CDR3, which comprises an amino acid sequence selected from the group consisting of: (i) SEQ ID NO. 31-40 and 136-141, and (ii) an amino acid sequence with one or more amino acid alterations as compared to SEQ ID NO. 31-40 and 136-141.

In some embodiments of the antibody of the present application, the antibody comprises: (1) a CDR1 comprising an amino acid sequence of SEQ ID NO. 11 or an amino acid sequence with one or more amino acid alterations as compared to SEQ ID NO. 11, (2) a CDR2 comprising an amino acid sequence of SEQ ID NO. 21 or an amino acid sequence with one or more amino acid alterations as compared to SEQ ID NO. 21, and (3) a CDR3 comprising an amino acid sequence of SEQ ID NO. 31 or an amino acid sequence with one or more amino acid alterations as compared to SEQ ID NO. 31.

In some embodiments of the antibody of the present application, the antibody comprises: (1) a CDR1 comprising an amino acid sequence of SEQ ID NO. 12 or an amino acid sequence with one or more amino acid alterations as compared to SEQ ID NO. 12, (2) a CDR2 comprising an amino acid sequence of SEQ ID NO. 22 or an amino acid sequence with one or more amino acid alterations as compared to SEQ ID NO. 22, and (3) a CDR3 comprising an amino acid sequence of SEQ ID NO. 32 or an amino acid sequence with one or more amino acid alterations as compared to SEQ ID NO. 32.

In some embodiments of the antibody of the present application, the antibody comprises: (1) a CDR1 comprising an amino acid sequence of SEQ ID NO. 13 or an amino acid sequence with one or more amino acid alterations as compared to SEQ ID NO. 13, (2) a CDR2 comprising an amino acid sequence of SEQ ID NO. 23 or an amino acid sequence with one or more amino acid alterations as compared to SEQ ID NO. 23, and (3) a CDR3 comprising an amino acid sequence of SEQ ID NO. 33 or an amino acid sequence with one or more amino acid alterations as compared to SEQ ID NO. 33.

In some embodiments of the antibody of the present application, the antibody comprises: (1) a CDR1 comprising an amino acid sequence of SEQ ID NO. 14 or an amino acid sequence with one or more amino acid alterations as compared to SEQ ID NO. 14, (2) a CDR2 comprising an amino acid sequence of SEQ ID NO. 24 or an amino acid sequence with one or more amino acid alterations as compared to SEQ ID NO. 24, and (3) a CDR3 comprising an amino acid sequence of SEQ ID NO. 34 or an amino acid sequence with one or more amino acid alterations as compared to SEQ ID NO. 34.

In some embodiments of the antibody of the present application, the antibody comprises: (1) a CDR1 comprising an amino acid sequence of SEQ ID NO. 15 or an amino acid sequence with one or more amino acid alterations as compared to SEQ ID NO. 15, (2) a CDR2 comprising an amino acid sequence of SEQ ID NO. 25 or an amino acid sequence with one or more amino acid alterations as compared to SEQ ID NO. 25, and (3) a CDR3 comprising an amino acid sequence of SEQ ID NO. 35 or an amino acid sequence with one or more amino acid alterations as compared to SEQ ID NO. 35.

In some embodiments of the antibody of the present application, the antibody comprises: (1) a CDR1 comprising an amino acid sequence of SEQ ID NO. 16 or an amino acid sequence with one or more amino acid alterations as compared to SEQ ID NO. 16, (2) a CDR2 comprising an amino acid sequence of SEQ ID NO. 26 or an amino acid sequence with one or more amino acid alterations as compared to SEQ ID NO. 26, and (3) a CDR3 comprising an amino acid sequence of SEQ ID NO. 36 or an amino acid sequence with one or more amino acid alterations as compared to SEQ ID NO. 36.

In some embodiments of the antibody of the present application, the antibody comprises: (1) a CDR1 comprising an amino acid sequence of SEQ ID NO. 17 or an amino acid sequence with one or more amino acid alterations as compared to SEQ ID NO. 17, (2) a CDR2 comprising an amino acid sequence of SEQ ID NO. 27 or an amino acid sequence with one or more amino acid alterations as compared to SEQ ID NO. 27, and (3) a CDR3 comprising an amino acid sequence of SEQ ID NO. 37 or an amino acid sequence with one or more amino acid alterations as compared to SEQ ID NO. 37.

In some embodiments of the antibody of the present application, the antibody comprises: (1) a CDR1 comprising an amino acid sequence of SEQ ID NO. 18 or an amino acid sequence with one or more amino acid alterations as compared to SEQ ID NO. 18, (2) a CDR2 comprising an amino acid sequence of SEQ ID NO. 28 or an amino acid sequence with one or more amino acid alterations as compared to SEQ ID NO. 28, and (3) a CDR3 comprising an amino acid sequence of SEQ ID NO. 38 or an amino acid sequence with one or more amino acid alterations as compared to SEQ ID NO. 38.

In some embodiments of the antibody of the present application, the antibody comprises: (1) a CDR1 comprising an amino acid sequence of SEQ ID NO. 19 or an amino acid sequence with one or more amino acid alterations as compared to SEQ ID NO. 19, (2) a CDR2 comprising an amino acid sequence of SEQ ID NO. 29 or an amino acid sequence with one or more amino acid alterations as compared to SEQ ID NO. 29, and (3) a CDR3 comprising an amino acid sequence of SEQ ID NO. 39 or an amino acid sequence with one or more amino acid alterations as compared to SEQ ID NO. 39.

In some embodiments of the antibody of the present application, the antibody comprises: (1) a CDR1 comprising an amino acid sequence of SEQ ID NO. 20 or an amino acid sequence with one or more amino acid alterations as compared to SEQ ID NO. 20, (2) a CDR2 comprising an amino acid sequence of SEQ ID NO. 30 or an amino acid sequence with one or more amino acid alterations as compared to SEQ ID NO. 30, and (3) a CDR3 comprising an amino acid sequence of SEQ ID NO. 40 or an amino acid sequence with one or more amino acid alterations as compared to SEQ ID NO. 40.

In some embodiments of the antibody of the present application, the antibody comprises: (1) a CDR1 comprising an amino acid sequence of SEQ ID NO. 124 or an amino acid sequence with one or more amino acid alterations as compared to SEQ ID NO. 124, (2) a CDR2 comprising an amino acid sequence of SEQ ID NO. 130 or an amino acid sequence with one or more amino acid alterations as compared to SEQ ID NO. 130, and (3) a CDR3 comprising an amino acid sequence of SEQ ID NO. 136 or an amino acid sequence with one or more amino acid alterations as compared to SEQ ID NO. 136.

In some embodiments of the antibody of the present application, the antibody comprises: (1) a CDR1 comprising an amino acid sequence of SEQ ID NO. 125 or an amino acid sequence with one or more amino acid alterations as compared to SEQ ID NO. 125, (2) a CDR2 comprising an amino acid sequence of SEQ ID NO. 131 or an amino acid sequence with one or more amino acid alterations as compared to SEQ ID NO. 131, and (3) a CDR3 comprising an amino acid sequence of SEQ ID NO. 137 or an amino acid sequence with one or more amino acid alterations as compared to SEQ ID NO. 137.

In some embodiments of the antibody of the present application, the antibody comprises: (1) a CDR1 comprising an amino acid sequence of SEQ ID NO. 126 or an amino acid sequence with one or more amino acid alterations as compared to SEQ ID NO. 126, (2) a CDR2 comprising an amino acid sequence of SEQ ID NO. 132 or an amino acid sequence with one or more amino acid alterations as compared to SEQ ID NO. 132, and (3) a CDR3 comprising an amino acid sequence of SEQ ID NO. 138 or an amino acid sequence with one or more amino acid alterations as compared to SEQ ID NO. 138.

In some embodiments of the antibody of the present application, the antibody comprises: (1) a CDR1 comprising an amino acid sequence of SEQ ID NO. 127 or an amino acid sequence with one or more amino acid alterations as compared to SEQ ID NO. 127, (2) a CDR2 comprising an amino acid sequence of SEQ ID NO. 133 or an amino acid sequence with one or more amino acid alterations as compared to SEQ ID NO. 133, and (3) a CDR3 comprising an amino acid sequence of SEQ ID NO. 139 or an amino acid sequence with one or more amino acid alterations as compared to SEQ ID NO. 139.

In some embodiments of the antibody of the present application, the antibody comprises: (1) a CDR1 comprising an amino acid sequence of SEQ ID NO. 128 or an amino acid sequence with one or more amino acid alterations as compared to SEQ ID NO. 128, (2) a CDR2 comprising an amino acid sequence of SEQ ID NO. 134 or an amino acid sequence with one or more amino acid alterations as compared to SEQ ID NO. 134, and (3) a CDR3 comprising an amino acid sequence of SEQ ID NO. 140 or an amino acid sequence with one or more amino acid alterations as compared to SEQ ID NO. 140.

In some embodiments of the antibody of the present application, the antibody comprises: (1) a CDR1 comprising an amino acid sequence of SEQ ID NO. 129 or an amino acid sequence with one or more amino acid alterations as compared to SEQ ID NO. 129, (2) a CDR2 comprising an amino acid sequence of SEQ ID NO. 135 or an amino acid sequence with one or more amino acid alterations as compared to SEQ ID NO. 135, and (3) a CDR3 comprising an amino acid sequence of SEQ ID NO. 141 or an amino acid sequence with one or more amino acid alterations as compared to SEQ ID NO. 141.

In some embodiments of the antibody of the present application, the antibody comprises an amino acid sequence having at least 70% identity, at least 80% identity, at least 90% identity, at least 95% identity, or at least 99% identity with anyone selected from the group consisting of SEQ ID NO. 1-10 and 82-123. In some embodiments of the antibody of the present application, the antibody comprises an amino acid sequence of anyone selected from the group consisting of SEQ ID NO. 1-10 and 82-123.

In another aspect, provided herein is an antibody construct, comprising the antibody as described herein. In some embodiments, the antibody construct further comprises a Fc fragment. In some embodiments, the Fc fragment is selected from the group consisting of IgG1, IgG2, IgG3 and IgG4. In some embodiments, the antibody construct is a multi-specific. In some embodiments, the antibody construct is bispecific.

In another aspect, provided herein is nucleic acid encoding the antibody or the antibody construct as described herein. In some embodiments, the nucleic acid comprises a polynucleotide sequence having at least 70%, at least 80%, at least 90%, at least 95% or at least 99% identity with anyone selected from the group consisting of SEQ ID NO. 41-50 and 160-165. In some embodiments, the nucleic acid comprises a polynucleotide sequence of anyone selected from the group consisting of SEQ ID NO. 41-50 and 160-165.

In another aspect, provided herein is a vector comprising the nucleic acid as described herein.

In another aspect, provided herein is a host cell comprising the nucleic acid or the vector as described herein.

In another aspect, provided herein is a conjugate comprising: (1) the antibody or the antibody construct as described herein, and (2) a drug. In some embodiments, the drug is selected from the group consisting of chemotherapeutic agent, toxin, cytokine, enzyme, immunomodulator, chelator, diagnostic agent, nanoparticle, and radioisotope. In some embodiments, the conjugate further comprises a linker.

In another aspect, provided herein is a chimeric antigen receptor (CAR) comprising the antibody construct as described herein. In another aspect, provided herein is a T-cell receptor (TCR) comprising the antibody construct as described herein.

In another aspect, provided herein is a cell expressing the CAR or the TCR as described herein.

In another aspect, provided herein is a pharmaceutical composition, comprising: (1) the antibody, the antibody construct, the nucleic acid, the conjugate or the cell as described herein, and (2) a pharmaceutically acceptable excipient.

In another aspect, provided herein is a method for treating a disorder in a subject in need thereof, comprising administrating an effective amount of the pharmaceutical composition as described herein to the subject. In another aspect, provided herein is a method for treating a disorder associated with the undesired presence of GCC+ cells, comprising administrating an effective amount of the pharmaceutical composition as described herein to the subject. In some embodiments, the disorder is cancer. In another aspect, provided herein is use of the pharmaceutical composition as described herien in preparing a medicament for treating a disorder in a subject in need thereof. In another aspect, provided herein is use of the antibody as described herein in detecting GCC in a sample from a subject.

The disclosures of all publications, patents, patent applications and published patent applications referred to herein are hereby incorporated herein by reference in their entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates binding affinity of exemplary anti-GCC antibodies of the present application by SPR.

FIG. 2 illustrates internalization of exemplary anti-GCC antibodies of the present application.

### DETAILED DESCRIPTION OF THE APPLICATION

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (dsdiabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “single-domain antibody (sdAb, also known as nanobody)” as used herein refers to an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (~50 kDa, one light chain and half a heavy chain) and single-chain variable fragments (~25 kDa, two variable domains, one from a light and one from a heavy chain).

The first single-domain antibodies were engineered from heavy-chain antibodies found in camelids; these are called VHH fragments. Cartilaginous fishes also have heavy-chain antibodies (IgNAR, immunoglobulin new antigen receptor), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

Camelid nanobodies have been shown to be as specific as antibodies, and in some cases they are more robust. They are easily isolated using the same phage panning procedure used for antibodies, allowing them to be cultured in vitro in large concentrations. The smaller size and single domain make these antibodies easier to transform into bacterial cells for bulk production, making them ideal for research purposes.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Pluckthun, supra

The expression “variable-domain residue-numbering as in Kabat” or “amino-acidposition numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the CDR residues as herein defined.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004).

The term “Fc region” or “fragment crystallizable region” herein is used to define a Cterminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The Cterminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, the terms “specifically binds,” “specifically recognizing,” and “is specific for” refer to measurable and reproducible interactions, such as binding between an antibody and an antigen thereof, which is determinative of the presence of the target or antigen in the presence of a heterogeneous population of molecules, including biological molecules. For example, an antibody that specifically recognizes an antigen is the antibody that binds this antigen with greater affinity, avidity, more readily, and/or with greater duration than its bindings to other targets or antigens. In some embodiments, the extent of binding of antibody to an unrelated target or antigen is less than about 10% of the binding of the antibody to the antigen thereof as measured, e.g., by a radioimmunoassay (RIA). In some embodiments, an antibody that specifically binds the antigen thereof has a dissociation constant (K_{D}) of ≤10⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, said specific binding can include, but does not require exclusive binding. Binding specificity of the antibody can be determined experimentally by methods known in the art. Such methods comprise, but are not limited to, e.g., Western blots, ELISA-, RIA-, ECL-, IRMA-, EIA-, BIACORE™-tests and peptide scans.

An “isolated” nucleic acid molecule encoding a polypeptides or a cytokine as described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. In some embodiments, the isolated nucleic acid molecules encoding the antibody or antibody construct described herein is in a form other than in the form or setting in which it is found in nature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### Antibody Binding Affinity

Binding affinity or binding specificity of an antibody as described herein can be determined experimentally by methods known in the art. Such methods comprise, but are not limited to Western blots, ELISA-, RIA-, ECL-, IRMA-, EIA-, BIACORE™-tests and peptide scans.

In some embodiments, the K_{D} of the binding between the antibody (or the antibody construct) and the target antigen (e.g., GCC) is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and the target antigen (e.g., GCC is less than about any one of 10⁻⁷ M, 10⁻⁸ M, 10⁻⁹ M, 10⁻¹⁰ M, 10⁻¹¹ M, or 10⁻¹² M.

In some embodiments, the Kₒₙ of the binding between the antibody (or the antibody construct) and the target antigen (e.g., GCC) is about 10³ M⁻¹s⁻¹ to about 10⁸ M⁻¹s⁻¹, about 10³ M-1s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and the target antigen (e.g., GCC) is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and the target antigen (e.g., GCC) is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹.

In some embodiments, the K_{off} of the binding between the antibody (or the antibody construct) and the target antigen (e.g., GCC) is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁴ s⁻¹ to about 10⁻⁶ s⁻¹, about 10⁻² s⁻¹ to about 10⁻⁵ s⁻¹, or about 10⁻³ s⁻¹ to about 10⁵ s⁻¹. In some embodiments, the K_{off} of the binding between the antibody moiety and the target antigen (e.g., GCC) is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹.

### Chimeric or Humanized Antibodies

In some embodiments, one or more of the antibodies of the present application is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigenbinding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (α-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Substitution, Insertion, and Deletion Variants

In some embodiments, antibody variants comprising one or more amino acid substitutions are included in the antibody described herein. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g.,retained/improved antigen binding, decreased immunogenicity, or improved ADCC or CDC.

TABLE 2
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

### Cysteine Engineered Antibody Variants

In some embodiments, it may be desirable to create cysteine engineered antibody moieties, e.g., “thioMAbs,” in which one or more residues of one or more of the antibody moieties in a multispecific construct herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibody moieties may be generated as described, e.g., in U.S. Pat. No. 7,521,541.

### Anti-GCC Single Domain Antibody

Guanylyl cyclase C (GCC) (also known as STAR, ST Receptor, GUC2C, and GUCY2C) is a transmembrane cell surface receptor which functions in the maintenance of intestinal fluid, electrolyte homeostasis and cell proliferation. It is expressed at the mucosal cells, e.g., at the apical brush border membranes, lining the small intestine, large intestine and rectum, and the expression is maintained upon neoplastic transformation of intestinal epithelial cells, with expression in all primary and metastatic colorectal tumors. Neoplastic cells from the stomach, esophagus and the gastroesophageal junction also express GCC, which is considered as a potential therapeutic target for receptor binding proteins such as antibodies or ligands. GCC protein comprises a signal sequence, an extracellular domain for ligand, e.g., guanylin or heat-stable enterotoxin (ST), a transmembrane domain, a kinase homology domain, and a guanylyl cyclase catalytic domain. In some embodiments, the GCC protein as described herein is a wild-type GCC. In some embodiments, the GCC protein is a natural variant GCC. In some embodiments, the GCC protein is a mutant GCC. In some embodiments, the GCC protein is a human GCC, such as comprising the amino acid sequence as set forth in SEQ ID NO. 81.

(SEQ ID NO. 81)
SQVSQNCHNGSYEISVLMMGNSAFAEPLKNLEDAVNEGLEIVRGRLQNAG
LNVTVNATFMYSDGLIHNSGDCRSSTCEGLDLLRKISNAQRMGCVLIGPS
CTYSTFQMYLDTELSYPMISAGSFGLSCDYKETLTRLMSPARKLMYFLVN
FWKTNDLPFKTYSWSTSYVYKNGTETEDCFWYLNALEASVSYFSHELGFK
VVLRQDKEFQDILMDHNRKSNVIIMCGGPEFLYKLKGDRAVAEDIVIILV
DLFNDQYFEDNVTAPDYMKNVLVLTLSPGNSLLNSSFSRNLSPTKRDFAL
AYLNGILLFGHMLKIFLENGENITTPKFAHAFRNLTFEGYDGPVTLDDWG
DVDSTMVLLYTSVDTKKYKVLLTYDTHVNKTYPVDMSPTFTWKNSKLPND
ITGRGPQ

In one aspect, provided herein is antibody specifically binding to guanylyl cyclase C (GCC) comprising CDR1, CDR2 and CDR3 of a single domain antibody of anyone selected from the group consisting of SEQ ID NO. 1-10 and 118-123. In some embodiments, the CDR1, CDR2 and CDR3 are according to IMGT numbering. In some embodiments, the CDR1, CDR2 and CDR3 are according to Kabat numbering. In some embodiments, the CDR1, CDR2 and CDR3 are according to Chothia numbering. In some embodiments, the CDR1, CDR2 and CDR3 are according to Abm numbering. In some embodiments, the CDR1, CDR2 and CDR3 are according to Contact numbering.

In another aspect of the present application, provided herein is antibody specifically binding to guanylyl cyclase C (GCC) comprising CDR1, CDR2 and CDR3 of a single domain antibody of anyone selected from the group consisting of SEQ ID NO. 1-10 and 118-123, and CDR1, CDR2 and CDR3 are according to IMGT numbering. In some embodiments, the the antibody comprises: (1) a CDR1, which comprises an amino acid sequence selected from the group consisting of: (i) SEQ ID NO. 11-20 and 124-129, and (ii) an amino acid sequence with one or more amino acid alterations (e.g., substitutions, deletions, or insertions) as compared to SEQ ID NO. 11-20 and 124-129; (2) a CDR2, which comprises an amino acid sequence selected from the group consisting of: (i) SEQ ID NO. 21-30 and 130-135, and (ii) an amino acid sequence with one or more amino acid alterations (e.g., substitutions, deletions, or insertions) as compared to SEQ ID NO. 21-30 and 130-135; and (3) a CDR3, which comprises an amino acid sequence selected from the group consisting of: (i) SEQ ID NO. 31-40 and 136-141, and (ii) an amino acid sequence with one or more amino acid alterations (e.g., substitutions, deletions, or insertions) as compared to SEQ ID NO. 31-40 and 136-141.

In some embodiments of the present application, provided herein is antibody specifically binding to guanylyl cyclase C (GCC) comprising CDR1, CDR2 and CDR3 of a single domain antibody of SEQ ID NO. 1. In some embodiments of the present application, provided herein is antibody specifically binding to guanylyl cyclase C (GCC) comprising CDR1, CDR2 and CDR3 of a single domain antibody of SEQ ID NO. 1, and the CDR1, CDR2 and CDR3 are according to IMGT numbering. In some embodiments, the antibody comprises: (1) a CDR1 comprising an amino acid sequence of SEQ ID NO. 11 or an amino acid sequence with one or more amino acid alterations (e.g., substitutions, deletions, or insertions) as compared to SEQ ID NO. 11, (2) a CDR2 comprising an amino acid sequence of SEQ ID NO. 21 or an amino acid sequence with one or more amino acid alterations (e.g., substitutions, deletions, or insertions) as compared to SEQ ID NO. 21, and (3) a CDR3 comprising an amino acid sequence of SEQ ID NO. 31 or an amino acid sequence with one or more amino acid alterations (e.g., substitutions, deletions, or insertions) as compared to SEQ ID NO. 31. In some embodiments, the antibody comprises: (1) a CDR1 comprising an amino acid sequence of SEQ ID NO. 11, (2) a CDR2 comprising an amino acid sequence of SEQ ID NO. 21, and (3) a CDR3 comprising an amino acid sequence of SEQ ID NO. 31. In some embodiments of the present application, provided herein is antibody specifically binding to guanylyl cyclase C (GCC) comprising CDR1, CDR2 and CDR3 of a single domain antibody of SEQ ID NO. 1, and the CDR1, CDR2 and CDR3 are according to Kabat numbering. In some embodiments, the antibody comprises: (1) a CDR1 comprising an amino acid sequence of SEQ ID NO. 51 or an amino acid sequence with one or more amino acid alterations (e.g., substitutions, deletions, or insertions) as compared to SEQ ID NO. 51, (2) a CDR2 comprising an amino acid sequence of SEQ ID NO. 61 or an amino acid sequence with one or more amino acid alterations (e.g., substitutions, deletions, or insertions) as compared to SEQ ID NO. 61, and (3) a CDR3 comprising an amino acid sequence of SEQ ID NO. 71 or an amino acid sequence with one or more amino acid alterations (e.g., substitutions, deletions, or insertions) as compared to SEQ ID NO. 71. In some embodiments, the antibody comprises: (1) a CDR1 comprising an amino acid sequence of SEQ ID NO. 51, (2) a CDR2 comprising an amino acid sequence of SEQ ID NO. 61, and (3) a CDR3 comprising an amino acid sequence of SEQ ID NO. 71. In some embodiments, the antibody comprises an amino acid sequence having at least 70% identity, at least 80% identity, at least 90% identity, at least 95% identity, at least 96% identity, at least 97% identity, at least 98% identity, or at least 99% identity with SEQ ID NO. 1. In some embodiments, the antibody comprises an amino acid sequence of SEQ ID NO. 1.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 2. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 2, and CDR1, CDR2 and CDR3 are according to IMGT numbering. In some embodiments, the antibody comprises: (1) a CDR1 comprising an amino acid sequence of SEQ ID NO. 12 or an amino acid sequence with one or more amino acid alterations (e.g., substitutions, deletions, or insertions) as compared to SEQ ID NO. 12, (2) a CDR2 comprising an amino acid sequence of SEQ ID NO. 22 or an amino acid sequence with one or more amino acid alterations (e.g., substitutions, deletions, or insertions) as compared to SEQ ID NO. 22, and (3) a CDR3 comprising an amino acid sequence of SEQ ID NO. 32 or an amino acid sequence with one or more amino acid alterations (e.g., substitutions, deletions, or insertions) as compared to SEQ ID NO. 32. In some embodiments, the antibody comprises: (1) a CDR1 comprising an amino acid sequence of SEQ ID NO. 12, (2) a CDR2 comprising an amino acid sequence of SEQ ID NO. 22, and (3) a CDR3 comprising an amino acid sequence of SEQ ID NO. 32. In some embodiments of the present application, provided herein is antibody specifically binding to guanylyl cyclase C (GCC) comprising CDR1, CDR2 and CDR3 of a single domain antibody of SEQ ID NO. 2, and the CDR1, CDR2 and CDR3 are according to Kabat numbering. In some embodiments, the antibody comprises: (1) a CDR1 comprising an amino acid sequence of SEQ ID NO. 52 or an amino acid sequence with one or more amino acid alterations (e.g., substitutions, deletions, or insertions) as compared to SEQ ID NO. 52, (2) a CDR2 comprising an amino acid sequence of SEQ ID NO. 62 or an amino acid sequence with one or more amino acid alterations (e.g., substitutions, deletions, or insertions) as compared to SEQ ID NO. 62, and (3) a CDR3 comprising an amino acid sequence of SEQ ID NO. 72 or an amino acid sequence with one or more amino acid alterations (e.g., substitutions, deletions, or insertions) as compared to SEQ ID NO. 72. In some embodiments, the antibody comprises: (1) a CDR1 comprising an amino acid sequence of SEQ ID NO. 52, (2) a CDR2 comprising an amino acid sequence of SEQ ID NO. 62, and (3) a CDR3 comprising an amino acid sequence of SEQ ID NO. 72. In some embodiments, the antibody comprises an amino acid with SEQ ID NO. 2. In some embodiments, the antibody comprises an amino acid sequence of SEQ ID NO. 2.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 3. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 3, and CDR1, CDR2 and CDR3 are according to IMGT numbering. In some embodiments, the antibody comprises: (1) a CDR1 comprising an amino acid sequence of SEQ ID NO. 13 or an amino acid sequence with one or more amino acid alterations (e.g., substitutions, deletions, or insertions) as compared to SEQ ID NO. 13, (2) a CDR2 comprising an amino acid sequence of SEQ ID NO. 23 or an amino acid sequence with one or more amino acid alterations (e.g., substitutions, deletions, or insertions) as compared to SEQ ID NO. 23, and (3) a CDR3 comprising an amino acid sequence of SEQ ID NO. 33 or an amino acid sequence with one or more amino acid alterations (e.g., substitutions, deletions, or insertions) as compared to SEQ ID NO. 33. In some embodiments, the antibody comprises: (1) a CDR1 comprising an amino acid sequence of SEQ ID NO. 13, (2) a CDR2 comprising an amino acid sequence of SEQ ID NO. 23, and (3) a CDR3 comprising an amino acid sequence of SEQ ID NO. 33. In some embodiments of the present application, provided herein is antibody specifically binding to guanylyl cyclase C (GCC) comprising CDR1, CDR2 and CDR3 of a single domain antibody of SEQ ID NO. 3, and the CDR1, CDR2 and CDR3 are according to Kabat numbering. In some embodiments, the antibody comprises: (1) a CDR1 comprising an amino acid sequence of SEQ ID NO. 53 or an amino acid sequence with one or more amino acid alterations (e.g., substitutions, deletions, or insertions) as compared to SEQ ID NO. 53, (2) a CDR2 comprising an amino acid sequence of SEQ ID NO. 63 or an amino acid sequence with one or more amino acid alterations (e.g., substitutions, deletions, or insertions) as compared to SEQ ID NO. 63, and (3) a CDR3 comprising an amino acid sequence of SEQ ID NO. 73 or an amino acid sequence with one or more amino acid alterations (e.g., substitutions, deletions, or insertions) as compared to SEQ ID NO. 73. In some embodiments, the antibody comprises: (1) a CDR1 comprising an amino acid sequence of SEQ ID NO. 53, (2) a CDR2 comprising an amino acid sequence of SEQ ID NO. 63, and (3) a CDR3 comprising an amino acid sequence of SEQ ID NO. 73. In some embodiments, the antibody comprises an amino acid with SEQ ID NO. 3. In some embodiments, the antibody comprises an amino acid sequence of SEQ ID NO. 3.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 4. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 4, and CDR1, CDR2 and CDR3 are according to IMGT numbering. In some embodiments, the antibody comprises: (1) a CDR1 comprising an amino acid sequence of SEQ ID NO. 14 or an amino acid sequence with one or more amino acid alterations (e.g., substitutions, deletions, or insertions) as compared to SEQ ID NO. 14, (2) a CDR2 comprising an amino acid sequence of SEQ ID NO. 24 or an amino acid sequence with one or more amino acid alterations (e.g., substitutions, deletions, or insertions) as compared to SEQ ID NO. 24, and (3) a CDR3 comprising an amino acid sequence of SEQ ID NO. 34 or an amino acid sequence with one or more amino acid alterations (e.g., substitutions, deletions, or insertions) as compared to SEQ ID NO. 34. In some embodiments, the antibody comprises: (1) a CDR1 comprising an amino acid sequence of SEQ ID NO. 14, (2) a CDR2 comprising an amino acid sequence of SEQ ID NO. 24, and (3) a CDR3 comprising an amino acid sequence of SEQ ID NO. 34. In some embodiments of the present application, provided herein is antibody specifically binding to guanylyl cyclase C (GCC) comprising CDR1, CDR2 and CDR3 of a single domain antibody of SEQ ID NO. 4, and the CDR1, CDR2 and CDR3 are according to Kabat numbering. In some embodiments, the antibody comprises: (1) a CDR1 comprising an amino acid sequence of SEQ ID NO. 54 or an amino acid sequence with one or more amino acid alterations (e.g., substitutions, deletions, or insertions) as compared to SEQ ID NO. 54, (2) a CDR2 comprising an amino acid sequence of SEQ ID NO. 64 or an amino acid sequence with one or more amino acid alterations (e.g., substitutions, deletions, or insertions) as compared to SEQ ID NO. 64, and (3) a CDR3 comprising an amino acid sequence of SEQ ID NO. 74 or an amino acid sequence with one or more amino acid alterations (e.g., substitutions, deletions, or insertions) as compared to SEQ ID NO. 74. In some embodiments, the antibody comprises: (1) a CDR1 comprising an amino acid sequence of SEQ ID NO. 54, (2) a CDR2 comprising an amino acid sequence of SEQ ID NO. 64, and (3) a CDR3 comprising an amino acid sequence of SEQ ID NO. 74. In some embodiments, the antibody comprises an amino acid with SEQ ID NO. 4. In some embodiments, the antibody comprises an amino acid sequence of SEQ ID NO. 4.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 5. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 5, and CDR1, CDR2 and CDR3 are according to IMGT numbering. In some embodiments, the antibody comprises: (1) a CDR1 comprising an amino acid sequence of SEQ ID NO. 15 or an amino acid sequence with one or more amino acid alterations (e.g., substitutions, deletions, or insertions) as compared to SEQ ID NO. 15, (2) a CDR2 comprising an amino acid sequence of SEQ ID NO. 25 or an amino acid sequence with one or more amino acid alterations (e.g., substitutions, deletions, or insertions) as compared to SEQ ID NO. 25, and (3) a CDR3 comprising an amino acid sequence of SEQ ID NO. 35 or an amino acid sequence with one or more amino acid alterations (e.g., substitutions, deletions, or insertions) as compared to SEQ ID NO. 35. In some embodiments, the antibody comprises: (1) a CDR1 comprising an amino acid sequence of SEQ ID NO. 15, (2) a CDR2 comprising an amino acid sequence of SEQ ID NO. 25, and (3) a CDR3 comprising an amino acid sequence of SEQ ID NO. 35. In some embodiments of the present application, provided herein is antibody specifically binding to guanylyl cyclase C (GCC) comprising CDR1, CDR2 and CDR3 of a single domain antibody of SEQ ID NO. 5, and the CDR1, CDR2 and CDR3 are according to Kabat numbering. In some embodiments, the antibody comprises: (1) a CDR1 comprising an amino acid sequence of SEQ ID NO. 55 or an amino acid sequence with one or more amino acid alterations (e.g., substitutions, deletions, or insertions) as compared to SEQ ID NO. 55, (2) a CDR2 comprising an amino acid sequence of SEQ ID NO. 65 or an amino acid sequence with one or more amino acid alterations (e.g., substitutions, deletions, or insertions) as compared to SEQ ID NO. 65, and (3) a CDR3 comprising an amino acid sequence of SEQ ID NO. 75 or an amino acid sequence with one or more amino acid alterations (e.g., substitutions, deletions, or insertions) as compared to SEQ ID NO. 75. In some embodiments, the antibody comprises: (1) a CDR1 comprising an amino acid sequence of SEQ ID NO. 55, (2) a CDR2 comprising an amino acid sequence of SEQ ID NO. 65, and (3) a CDR3 comprising an amino acid sequence of SEQ ID NO. 75. In some embodiments, the antibody comprises an amino acid with SEQ ID NO. 5. In some embodiments, the antibody comprises an amino acid sequence of SEQ ID NO. 5.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 6. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 6, and CDR1, CDR2 and CDR3 are according to IMGT numbering. In some embodiments, the antibody comprises: (1) a CDR1 comprising an amino acid sequence of SEQ ID NO. 16 or an amino acid sequence with one or more amino acid alterations (e.g., substitutions, deletions, or insertions) as compared to SEQ ID NO. 16, (2) a CDR2 comprising an amino acid sequence of SEQ ID NO. 26 or an amino acid sequence with one or more amino acid alterations (e.g., substitutions, deletions, or insertions) as compared to SEQ ID NO. 26, and (3) a CDR3 comprising an amino acid sequence of SEQ ID NO. 36 or an amino acid sequence with one or more amino acid alterations (e.g., substitutions, deletions, or insertions) as compared to SEQ ID NO. 36. In some embodiments, the antibody comprises: (1) a CDR1 comprising an amino acid sequence of SEQ ID NO. 16, (2) a CDR2 comprising an amino acid sequence of SEQ ID NO. 26, and (3) a CDR3 comprising an amino acid sequence of SEQ ID NO. 36. In some embodiments of the present application, provided herein is antibody specifically binding to guanylyl cyclase C (GCC) comprising CDR1, CDR2 and CDR3 of a single domain antibody of SEQ ID NO. 6, and the CDR1, CDR2 and CDR3 are according to Kabat numbering. In some embodiments, the antibody comprises: (1) a CDR1 comprising an amino acid sequence of SEQ ID NO. 56 or an amino acid sequence with one or more amino acid alterations (e.g., substitutions, deletions, or insertions) as compared to SEQ ID NO. 56, (2) a CDR2 comprising an amino acid sequence of SEQ ID NO. 66 or an amino acid sequence with one or more amino acid alterations (e.g., substitutions, deletions, or insertions) as compared to SEQ ID NO. 66, and (3) a CDR3 comprising an amino acid sequence of SEQ ID NO. 76 or an amino acid sequence with one or more amino acid alterations (e.g., substitutions, deletions, or insertions) as compared to SEQ ID NO. 76. In some embodiments, the antibody comprises: (1) a CDR1 comprising an amino acid sequence of SEQ ID NO. 56, (2) a CDR2 comprising an amino acid sequence of SEQ ID NO. 66, and (3) a CDR3 comprising an amino acid sequence of SEQ ID NO. 76. In some embodiments, the antibody comprises an amino acid with SEQ ID NO. 6. In some embodiments, the antibody comprises an amino acid sequence of SEQ ID NO. 6.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 7. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 7, and CDR1, CDR2 and CDR3 are according to IMGT numbering. In some embodiments, the antibody comprises: (1) a CDR1 comprising an amino acid sequence of SEQ ID NO. 17 or an amino acid sequence with one or more amino acid alterations (e.g., substitutions, deletions, or insertions) as compared to SEQ ID NO. 17, (2) a CDR2 comprising an amino acid sequence of SEQ ID NO. 27 or an amino acid sequence with one or more amino acid alterations (e.g., substitutions, deletions, or insertions) as compared to SEQ ID NO. 27, and (3) a CDR3 comprising an amino acid sequence of SEQ ID NO. 37 or an amino acid sequence with one or more amino acid alterations (e.g., substitutions, deletions, or insertions) as compared to SEQ ID NO. 37. In some embodiments, the antibody comprises: (1) a CDR1 comprising an amino acid sequence of SEQ ID NO. 17, (2) a CDR2 comprising an amino acid sequence of SEQ ID NO. 27, and (3) a CDR3 comprising an amino acid sequence of SEQ ID NO. 37. In some embodiments of the present application, provided herein is antibody specifically binding to guanylyl cyclase C (GCC) comprising CDR1, CDR2 and CDR3 of a single domain antibody of SEQ ID NO. 7, and the CDR1, CDR2 and CDR3 are according to Kabat numbering. In some embodiments, the antibody comprises: (1) a CDR1 comprising an amino acid sequence of SEQ ID NO. 57 or an amino acid sequence with one or more amino acid alterations (e.g., substitutions, deletions, or insertions) as compared to SEQ ID NO. 57, (2) a CDR2 comprising an amino acid sequence of SEQ ID NO. 67 or an amino acid sequence with one or more amino acid alterations (e.g., substitutions, deletions, or insertions) as compared to SEQ ID NO. 67, and (3) a CDR3 comprising an amino acid sequence of SEQ ID NO. 77 or an amino acid sequence with one or more amino acid alterations (e.g., substitutions, deletions, or insertions) as compared to SEQ ID NO. 77. In some embodiments, the antibody comprises: (1) a CDR1 comprising an amino acid sequence of SEQ ID NO. 57, (2) a CDR2 comprising an amino acid sequence of SEQ ID NO. 67, and (3) a CDR3 comprising an amino acid sequence of SEQ ID NO. 77. In some embodiments, the antibody comprises an amino acid identity, at least 96% identity, at least 97% identity, at least 98% identity, or at least 99% identity with SEQ ID NO. 7. In some embodiments, the antibody comprises an amino acid sequence of SEQ ID NO. 7.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 8. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 8, and CDR1, CDR2 and CDR3 are according to IMGT numbering. In some embodiments, the antibody comprises: (1) a CDR1 comprising an amino acid sequence of SEQ ID NO. 18 or an amino acid sequence with one or more amino acid alterations (e.g., substitutions, deletions, or insertions) as compared to SEQ ID NO. 18, (2) a CDR2 comprising an amino acid sequence of SEQ ID NO. 28 or an amino acid sequence with one or more amino acid alterations (e.g., substitutions, deletions, or insertions) as compared to SEQ ID NO. 28, and (3) a CDR3 comprising an amino acid sequence of SEQ ID NO. 38 or an amino acid sequence with one or more amino acid alterations (e.g., substitutions, deletions, or insertions) as compared to SEQ ID NO. 38. In some embodiments, the antibody comprises: (1) a CDR1 comprising an amino acid sequence of SEQ ID NO. 18, (2) a CDR2 comprising an amino acid sequence of SEQ ID NO. 28, and (3) a CDR3 comprising an amino acid sequence of SEQ ID NO. 38. In some embodiments of the present application, provided herein is antibody specifically binding to guanylyl cyclase C (GCC) comprising CDR1, CDR2 and CDR3 of a single domain antibody of SEQ ID NO. 8, and the CDR1, CDR2 and CDR3 are according to Kabat numbering. In some embodiments, the antibody comprises: (1) a CDR1 comprising an amino acid sequence of SEQ ID NO. 58 or an amino acid sequence with one or more amino acid alterations (e.g., substitutions, deletions, or insertions) as compared to SEQ ID NO. 58, (2) a CDR2 comprising an amino acid sequence of SEQ ID NO. 68 or an amino acid sequence with one or more amino acid alterations (e.g., substitutions, deletions, or insertions) as compared to SEQ ID NO. 68, and (3) a CDR3 comprising an amino acid sequence of SEQ ID NO. 78 or an amino acid sequence with one or more amino acid alterations (e.g., substitutions, deletions, or insertions) as compared to SEQ ID NO. 78. In some embodiments, the antibody comprises: (1) a CDR1 comprising an amino acid sequence of SEQ ID NO. 58, (2) a CDR2 comprising an amino acid sequence of SEQ ID NO. 68, and (3) a CDR3 comprising an amino acid sequence of SEQ ID NO. 78. In some embodiments, the antibody comprises an amino acid with SEQ ID NO. 8. In some embodiments, the antibody comprises an amino acid sequence of SEQ ID NO. 8.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 9. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 9, and CDR1, CDR2 and CDR3 are according to IMGT numbering. In some embodiments, the antibody comprises: (1) a CDR1 comprising an amino acid sequence of SEQ ID NO. 19 or an amino acid sequence with one or more amino acid alterations (e.g., substitutions, deletions, or insertions) as compared to SEQ ID NO. 19, (2) a CDR2 comprising an amino acid sequence of SEQ ID NO. 29 or an amino acid sequence with one or more amino acid alterations (e.g., substitutions, deletions, or insertions) as compared to SEQ ID NO. 29, and (3) a CDR3 comprising an amino acid sequence of SEQ ID NO. 39 or an amino acid sequence with one or more amino acid alterations (e.g., substitutions, deletions, or insertions) as compared to SEQ ID NO. 39. In some embodiments, the antibody comprises: (1) a CDR1 comprising an amino acid sequence of SEQ ID NO. 19, (2) a CDR2 comprising an amino acid sequence of SEQ ID NO. 29, and (3) a CDR3 comprising an amino acid sequence of SEQ ID NO. 39. In some embodiments of the present application, provided herein is antibody specifically binding to guanylyl cyclase C (GCC) comprising CDR1, CDR2 and CDR3 of a single domain antibody of SEQ ID NO. 9, and the CDR1, CDR2 and CDR3 are according to Kabat numbering. In some embodiments, the antibody comprises: (1) a CDR1 comprising an amino acid sequence of SEQ ID NO. 59 or an amino acid sequence with one or more amino acid alterations (e.g., substitutions, deletions, or insertions) as compared to SEQ ID NO. 59, (2) a CDR2 comprising an amino acid sequence of SEQ ID NO. 69 or an amino acid sequence with one or more amino acid alterations (e.g., substitutions, deletions, or insertions) as compared to SEQ ID NO. 69, and (3) a CDR3 comprising an amino acid sequence of SEQ ID NO. 79 or an amino acid sequence with one or more amino acid alterations (e.g., substitutions, deletions, or insertions) as compared to SEQ ID NO. 79. In some embodiments, the antibody comprises: (1) a CDR1 comprising an amino acid sequence of SEQ ID NO. 59, (2) a CDR2 comprising an amino acid sequence of SEQ ID NO. 69, and (3) a CDR3 comprising an amino acid sequence of SEQ ID NO. 79. In some embodiments, the antibody comprises an amino acid with SEQ ID NO. 9. In some embodiments, the antibody comprises an amino acid sequence of SEQ ID NO. 9.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 10. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 10, and CDR1, CDR2 and CDR3 are according to IMGT numbering. In some embodiments, the antibody comprises: (1) a CDR1 comprising an amino acid sequence of SEQ ID NO. 20 or an amino acid sequence with one or more amino acid alterations (e.g., substitutions, deletions, or insertions) as compared to SEQ ID NO. 20, (2) a CDR2 comprising an amino acid sequence of SEQ ID NO. 30 or an amino acid sequence with one or more amino acid alterations (e.g., substitutions, deletions, or insertions) as compared to SEQ ID NO. 30, and (3) a CDR3 comprising an amino acid sequence of SEQ ID NO. 40 or an amino acid sequence with one or more amino acid alterations (e.g., substitutions, deletions, or insertions) as compared to SEQ ID NO. 40. In some embodiments, the antibody comprises: (1) a CDR1 comprising an amino acid sequence of SEQ ID NO. 20, (2) a CDR2 comprising an amino acid sequence of SEQ ID NO. 30, and (3) a CDR3 comprising an amino acid sequence of SEQ ID NO. 40. In some embodiments of the present application, provided herein is antibody specifically binding to guanylyl cyclase C (GCC) comprising CDR1, CDR2 and CDR3 of a single domain antibody of SEQ ID NO. 10, and the CDR1, CDR2 and CDR3 are according to Kabat numbering. In some embodiments, the antibody comprises: (1) a CDR1 comprising an amino acid sequence of SEQ ID NO. 60 or an amino acid sequence with one or more amino acid alterations (e.g., substitutions, deletions, or insertions) as compared to SEQ ID NO. 60, (2) a CDR2 comprising an amino acid sequence of SEQ ID NO. 70 or an amino acid sequence with one or more amino acid alterations (e.g., substitutions, deletions, or insertions) as compared to SEQ ID NO. 70, and (3) a CDR3 comprising an amino acid sequence of SEQ ID NO. 80 or an amino acid sequence with one or more amino acid alterations (e.g., substitutions, deletions, or insertions) as compared to SEQ ID NO. 80. In some embodiments, the antibody comprises: (1) a CDR1 comprising an amino acid sequence of SEQ ID NO. 60, (2) a CDR2 comprising an amino acid sequence of SEQ ID NO. 70, and (3) a CDR3 comprising an amino acid sequence of SEQ ID NO. 80. In some embodiments, the antibody comprises an amino acid with SEQ ID NO. 10. In some embodiments, the antibody comprises an amino acid sequence of SEQ ID NO. 10.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 118. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 118, and CDR1, CDR2 and CDR3 are according to IMGT numbering. In some embodiments, the antibody comprises: (1) a CDR1 comprising an amino acid sequence of SEQ ID NO. 124 or an amino acid sequence with one or more amino acid alterations (e.g., substitutions, deletions, or insertions) as compared to SEQ ID NO. 124, (2) a CDR2 comprising an amino acid sequence of SEQ ID NO. 130 or an amino acid sequence with one or more amino acid alterations (e.g., substitutions, deletions, or insertions) as compared to SEQ ID NO. 130, and (3) a CDR3 comprising an amino acid sequence of SEQ ID NO. 136 or an amino acid sequence with one or more amino acid alterations (e.g., substitutions, deletions, or insertions) as compared to SEQ ID NO. 136. In some embodiments, the antibody comprises: (1) a CDR1 comprising an amino acid sequence of SEQ ID NO. 124, (2) a CDR2 comprising an amino acid sequence of SEQ ID NO. 130, and (3) a CDR3 comprising an amino acid sequence of SEQ ID NO. 136. In some embodiments of the present application, provided herein is antibody specifically binding to guanylyl cyclase C (GCC) comprising CDR1, CDR2 and CDR3 of a single domain antibody of SEQ ID NO. 118, and the CDR1, CDR2 and CDR3 are according to Kabat numbering. In some embodiments, the antibody comprises: (1) a CDR1 comprising an amino acid sequence of SEQ ID NO. 142 or an amino acid sequence with one or more amino acid alterations (e.g., substitutions, deletions, or insertions) as compared to SEQ ID NO. 142, (2) a CDR2 comprising an amino acid sequence of SEQ ID NO. 148 or an amino acid sequence with one or more amino acid alterations (e.g., substitutions, deletions, or insertions) as compared to SEQ ID NO. 148, and (3) a CDR3 comprising an amino acid sequence of SEQ ID NO. 154 or an amino acid sequence with one or more amino acid alterations (e.g., substitutions, deletions, or insertions) as compared to SEQ ID NO. 154. In some embodiments, the antibody comprises: (1) a CDR1 comprising an amino acid sequence of SEQ ID NO. 142, (2) a CDR2 comprising an amino acid sequence of SEQ ID NO. 148, and (3) a CDR3 comprising an amino acid sequence of SEQ ID NO. 154. In some embodiments, the antibody comprises an amino identity with SEQ ID NO. 118. In some embodiments, the antibody comprises an amino acid sequence of SEQ ID NO. 118.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 119. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 119, and CDR1, CDR2 and CDR3 are according to IMGT numbering. In some embodiments, the antibody comprises: (1) a CDR1 comprising an amino acid sequence of SEQ ID NO. 125 or an amino acid sequence with one or more amino acid alterations (e.g., substitutions, deletions, or insertions) as compared to SEQ ID NO. 125, (2) a CDR2 comprising an amino acid sequence of SEQ ID NO. 131 or an amino acid sequence with one or more amino acid alterations (e.g., substitutions, deletions, or insertions) as compared to SEQ ID NO. 131, and (3) a CDR3 comprising an amino acid sequence of SEQ ID NO. 137 or an amino acid sequence with one or more amino acid alterations (e.g., substitutions, deletions, or insertions) as compared to SEQ ID NO. 137. In some embodiments, the antibody comprises: (1) a CDR1 comprising an amino acid sequence of SEQ ID NO. 125, (2) a CDR2 comprising an amino acid sequence of SEQ ID NO. 131, and (3) a CDR3 comprising an amino acid sequence of SEQ ID NO. 137. In some embodiments of the present application, provided herein is antibody specifically binding to guanylyl cyclase C (GCC) comprising CDR1, CDR2 and CDR3 of a single domain antibody of SEQ ID NO. 119, and the CDR1, CDR2 and CDR3 are according to Kabat numbering. In some embodiments, the antibody comprises: (1) a CDR1 comprising an amino acid sequence of SEQ ID NO. 142 or an amino acid sequence with one or more amino acid alterations (e.g., substitutions, deletions, or insertions) as compared to SEQ ID NO. 142, (2) a CDR2 comprising an amino acid sequence of SEQ ID NO. 148 or an amino acid sequence with one or more amino acid alterations (e.g., substitutions, deletions, or insertions) as compared to SEQ ID NO. 148, and (3) a CDR3 comprising an amino acid sequence of SEQ ID NO. 154 or an amino acid sequence with one or more amino acid alterations (e.g., substitutions, deletions, or insertions) as compared to SEQ ID NO. 154. In some embodiments, the antibody comprises: (1) a CDR1 comprising an amino acid sequence of SEQ ID NO. 142, (2) a CDR2 comprising an amino acid sequence of SEQ ID NO. 148, and (3) a CDR3 comprising an amino acid sequence of SEQ ID NO. 154. In some embodiments, the antibody comprises an amino identity with SEQ ID NO. 119. In some embodiments, the antibody comprises an amino acid sequence of SEQ ID NO. 119.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 120. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 120, and CDR1, CDR2 and CDR3 are according to IMGT numbering. In some embodiments, the antibody comprises: (1) a CDR1 comprising an amino acid sequence of SEQ ID NO. 126 or an amino acid sequence with one or more amino acid alterations (e.g., substitutions, deletions, or insertions) as compared to SEQ ID NO. 126, (2) a CDR2 comprising an amino acid sequence of SEQ ID NO. 132 or an amino acid sequence with one or more amino acid alterations (e.g., substitutions, deletions, or insertions) as compared to SEQ ID NO. 132, and (3) a CDR3 comprising an amino acid sequence of SEQ ID NO. 138 or an amino acid sequence with one or more amino acid alterations (e.g., substitutions, deletions, or insertions) as compared to SEQ ID NO. 138. In some embodiments, the antibody comprises: (1) a CDR1 comprising an amino acid sequence of SEQ ID NO. 126, (2) a CDR2 comprising an amino acid sequence of SEQ ID NO. 132, and (3) a CDR3 comprising an amino acid sequence of SEQ ID NO. 138. In some embodiments of the present application, provided herein is antibody specifically binding to guanylyl cyclase C (GCC) comprising CDR1, CDR2 and CDR3 of a single domain antibody of SEQ ID NO. 120, and the CDR1, CDR2 and CDR3 are according to Kabat numbering. In some embodiments, the antibody comprises: (1) a CDR1 comprising an amino acid sequence of SEQ ID NO. 144 or an amino acid sequence with one or more amino acid alterations (e.g., substitutions, deletions, or insertions) as compared to SEQ ID NO. 144, (2) a CDR2 comprising an amino acid sequence of SEQ ID NO. 150 or an amino acid sequence with one or more amino acid alterations (e.g., substitutions, deletions, or insertions) as compared to SEQ ID NO. 150, and (3) a CDR3 comprising an amino acid sequence of SEQ ID NO. 156 or an amino acid sequence with one or more amino acid alterations (e.g., substitutions, deletions, or insertions) as compared to SEQ ID NO. 156. In some embodiments, the antibody comprises: (1) a CDR1 comprising an amino acid sequence of SEQ ID NO. 144, (2) a CDR2 comprising an amino acid sequence of SEQ ID NO. 150, and (3) a CDR3 comprising an amino acid sequence of SEQ ID NO. 156. In some embodiments, the antibody comprises an amino identity with SEQ ID NO. 120. In some embodiments, the antibody comprises an amino acid sequence of SEQ ID NO. 120.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 121. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 121, and CDR1, CDR2 and CDR3 are according to IMGT numbering. In some embodiments, the antibody comprises: (1) a CDR1 comprising an amino acid sequence of SEQ ID NO. 127 or an amino acid sequence with one or more amino acid alterations (e.g., substitutions, deletions, or insertions) as compared to SEQ ID NO. 127, (2) a CDR2 comprising an amino acid sequence of SEQ ID NO. 133 or an amino acid sequence with one or more amino acid alterations (e.g., substitutions, deletions, or insertions) as compared to SEQ ID NO. 133, and (3) a CDR3 comprising an amino acid sequence of SEQ ID NO. 139 or an amino acid sequence with one or more amino acid alterations (e.g., substitutions, deletions, or insertions) as compared to SEQ ID NO. 139. In some embodiments, the antibody comprises: (1) a CDR1 comprising an amino acid sequence of SEQ ID NO. 127, (2) a CDR2 comprising an amino acid sequence of SEQ ID NO. 133, and (3) a CDR3 comprising an amino acid sequence of SEQ ID NO. 139. In some embodiments of the present application, provided herein is antibody specifically binding to guanylyl cyclase C (GCC) comprising CDR1, CDR2 and CDR3 of a single domain antibody of SEQ ID NO. 121, and the CDR1, CDR2 and CDR3 are according to Kabat numbering. In some embodiments, the antibody comprises: (1) a CDR1 comprising an amino acid sequence of SEQ ID NO. 145 or an amino acid sequence with one or more amino acid alterations (e.g., substitutions, deletions, or insertions) as compared to SEQ ID NO. 145, (2) a CDR2 comprising an amino acid sequence of SEQ ID NO. 151 or an amino acid sequence with one or more amino acid alterations (e.g., substitutions, deletions, or insertions) as compared to SEQ ID NO. 151, and (3) a CDR3 comprising an amino acid sequence of SEQ ID NO. 157 or an amino acid sequence with one or more amino acid alterations (e.g., substitutions, deletions, or insertions) as compared to SEQ ID NO. 157. In some embodiments, the antibody comprises: (1) a CDR1 comprising an amino acid sequence of SEQ ID NO. 145, (2) a CDR2 comprising an amino acid sequence of SEQ ID NO. 151, and (3) a CDR3 comprising an amino acid sequence of SEQ ID NO. 157. In some embodiments, the antibody comprises an amino identity with SEQ ID NO. 121. In some embodiments, the antibody comprises an amino acid sequence of SEQ ID NO. 121.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 122. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 122, and CDR1, CDR2 and CDR3 are according to IMGT numbering. In some embodiments, the antibody comprises: (1) a CDR1 comprising an amino acid sequence of SEQ ID NO. 128 or an amino acid sequence with one or more amino acid alterations (e.g., substitutions, deletions, or insertions) as compared to SEQ ID NO. 128, (2) a CDR2 comprising an amino acid sequence of SEQ ID NO. 134 or an amino acid sequence with one or more amino acid alterations (e.g., substitutions, deletions, or insertions) as compared to SEQ ID NO. 134, and (3) a CDR3 comprising an amino acid sequence of SEQ ID NO. 140 or an amino acid sequence with one or more amino acid alterations (e.g., substitutions, deletions, or insertions) as compared to SEQ ID NO. 140. In some embodiments, the antibody comprises: (1) a CDR1 comprising an amino acid sequence of SEQ ID NO. 128, (2) a CDR2 comprising an amino acid sequence of SEQ ID NO. 134, and (3) a CDR3 comprising an amino acid sequence of SEQ ID NO. 140. In some embodiments of the present application, provided herein is antibody specifically binding to guanylyl cyclase C (GCC) comprising CDR1, CDR2 and CDR3 of a single domain antibody of SEQ ID NO. 122, and the CDR1, CDR2 and CDR3 are according to Kabat numbering. In some embodiments, the antibody comprises: (1) a CDR1 comprising an amino acid sequence of SEQ ID NO. 146 or an amino acid sequence with one or more amino acid alterations (e.g., substitutions, deletions, or insertions) as compared to SEQ ID NO. 146, (2) a CDR2 comprising an amino acid sequence of SEQ ID NO. 152 or an amino acid sequence with one or more amino acid alterations (e.g., substitutions, deletions, or insertions) as compared to SEQ ID NO. 152, and (3) a CDR3 comprising an amino acid sequence of SEQ ID NO. 158 or an amino acid sequence with one or more amino acid alterations (e.g., substitutions, deletions, or insertions) as compared to SEQ ID NO. 158. In some embodiments, the antibody comprises: (1) a CDR1 comprising an amino acid sequence of SEQ ID NO. 146, (2) a CDR2 comprising an amino acid sequence of SEQ ID NO. 152, and (3) a CDR3 comprising an amino acid sequence of SEQ ID NO. 158. In some embodiments, the antibody comprises an amino identity with SEQ ID NO. 122. In some embodiments, the antibody comprises an amino acid sequence of SEQ ID NO. 122.

In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 123. In some embodiments of the present application, provided herein is antibody specifically binding to guanylyl cyclase C (GCC) comprising CDR1, CDR2 and CDR3 of a single domain antibody of anyone selected from the group consisting of SEQ ID NO. 123, and CDR1, CDR2 and CDR3 are according to IMGT numbering. In some embodiments, the antibody comprises: (1) a CDR1 comprising an amino acid sequence of SEQ ID NO. 129 or an amino acid sequence with one or more amino acid alterations (e.g., substitutions, deletions, or insertions) as compared to SEQ ID NO. 129, (2) a CDR2 comprising an amino acid sequence of SEQ ID NO. 135 or an amino acid sequence with one or more amino acid alterations (e.g., substitutions, deletions, or insertions) as compared to SEQ ID NO. 135, and (3) a CDR3 comprising an amino acid sequence of SEQ ID NO. 141 or an amino acid sequence with one or more amino acid alterations (e.g., substitutions, deletions, or insertions) as compared to SEQ ID NO. 141. In some embodiments, the antibody comprises: (1) a CDR1 comprising an amino acid sequence of SEQ ID NO. 129, (2) a CDR2 comprising an amino acid sequence of SEQ ID NO. 135, and (3) a CDR3 comprising an amino acid sequence of SEQ ID NO. 141. In some embodiments of the present application, provided herein is antibody specifically binding to guanylyl cyclase C (GCC) comprising CDR1, CDR2 and CDR3 of a single domain antibody of SEQ ID NO. 123, and the CDR1, CDR2 and CDR3 are according to Kabat numbering. In some embodiments, the antibody comprises: (1) a CDR1 comprising an amino acid sequence of SEQ ID NO. 147 or an amino acid sequence with one or more amino acid alterations (e.g., substitutions, deletions, or insertions) as compared to SEQ ID NO. 147, (2) a CDR2 comprising an amino acid sequence of SEQ ID NO. 153 or an amino acid sequence with one or more amino acid alterations (e.g., substitutions, deletions, or insertions) as compared to SEQ ID NO. 153, and (3) a CDR3 comprising an amino acid sequence of SEQ ID NO. 159 or an amino acid sequence with one or more amino acid alterations (e.g., substitutions, deletions, or insertions) as compared to SEQ ID NO. 159. In some embodiments, the antibody comprises: (1) a CDR1 comprising an amino acid sequence of SEQ ID NO.147, (2) a CDR2 comprising an amino acid sequence of SEQ ID NO. 153, and (3) a CDR3 comprising an amino acid sequence of SEQ ID NO. 159. In some embodiments, the antibody comprises an amino identity with SEQ ID NO. 123. In some embodiments, the antibody comprises an amino acid sequence of SEQ ID NO. 123.

In some embodiments of the antibody of the present application, the antibody is a humanized antibody. In some embodiments, the humanized antibody is derived from anyone selected from the group consisting of SEQ ID NO. 1-10 and 118-123.

In some embodiments, the humanized antibody is derived from SEQ ID NO. 1 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 82-86. In some embodiments, the humanized antibody is derived from SEQ ID NO. 1 and comprises an amino acid sequence of any one selected from the group consisting of SEQ ID NO. 82-86.

In some embodiments, the humanized antibody is derived from SEQ ID NO. 2 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 87-90. In some embodiments, the humanized antibody is derived from SEQ ID NO. 2 and comprises an amino acid sequence of any one selected from the group consisting of SEQ ID NO. 87-90.

In some embodiments, the humanized antibody is derived from SEQ ID NO. 3 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 91-94. In some embodiments, the humanized antibody is derived from SEQ ID NO. 3 and comprises an amino acid sequence of any one selected from the group consisting of SEQ ID NO. 91-94.

In some embodiments, the humanized antibody is derived from SEQ ID NO. 4 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 95-98. In some embodiments, the humanized antibody is derived from SEQ ID NO. 4 and comprises an amino acid sequence of any one selected from the group consisting of SEQ ID NO. 95-98.

In some embodiments, the humanized antibody is derived from SEQ ID NO. 5 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 99-101. In some embodiments, the humanized antibody is derived from SEQ ID NO. 5 and comprises an amino acid sequence of any one selected from the group consisting of SEQ ID NO. 99-101.

In some embodiments, the humanized antibody is derived from SEQ ID NO. 6 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 102-10⁵. In some embodiments, the humanized antibody is derived from SEQ ID NO. 6 and comprises an amino acid sequence of any one selected from the group consisting of SEQ ID NO. 102-10⁵.

In some embodiments, the humanized antibody is derived from SEQ ID NO. 7 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 106-110. In some embodiments, the humanized antibody is derived from SEQ ID NO. 7 and comprises an amino acid sequence of any one selected from the group consisting of SEQ ID NO. 106-110.

In some embodiments, the humanized antibody is derived from SEQ ID NO. 8 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 111-114. In some embodiments, the humanized antibody is derived from SEQ ID NO. 8 and comprises an amino acid sequence of any one selected from the group consisting of SEQ ID NO. 111-114.

In some embodiments, the humanized antibody is derived from SEQ ID NO. 9 and comprises an amino acid sequence having at least 70% identity, at least 80% identity, at least 90% identity, at least 95% identity, at least 96% identity, at least 97% identity, at least 98% identity, or at least 99% identity with anyone selected from the group consisting of SEQ ID NO. 115-117. In some embodiments, the humanized antibody is derived from SEQ ID NO. 9 and comprises an amino acid sequence of any one selected from the group consisting of SEQ ID NO. 115-117.

In some embodiments of the antibody of the present application, the antibody is a single domain antibody.

In another aspect, provided herein is an antibody construct comprising the antibody as described herein. In some embodiments, the antibody construct further comprises one or more additional antibody moieties. In some embodiments, the antibody construct is a multispecific antibody capable of specifically binding to GCC and one or more additional targets. In some embodiments, the antibody construct is a bispecific antibody capable of specifically binding to GCC and a second target.

The term “bispecific” as used herein refers to an antibody with one part of the antibody binds to one epitope on an antigen whereas the second part binds to a different epitope on the antigen, or on a different antigen. The different epitope is typically present on a different antigen. In some embodiments, the bispecific antibody is an antibody that comprises parts of two different antibodies and consequently binds to two different types of antigen. One arm of the bispecific antibody typically contains a variable domain of one antibody and the other arm contains a variable domain of another antibody. In some embodiments, one arm of the bispecific antibody typically contains a variable domain targeting GCC and the other arm contains a variable domain of another antibody targeting the second target. In some embodiments, one arm of the bispecific antibody typically contains the single domain antibody specifically binding to GCC as described herein and the other arm contains a variable domain of another antibody targeting the second target.

In some embodiments, the second target is a tumor antigen. In some embodiments, the tumor antigen can be selected from the group consisting of CD2, CD3, CD4, CD5, CD7, CD8, CD19, CD20, CD22, CD25, CD28, CD30, CD33, CD38, CD40, CD44V6, CD47, CD52, CD56, CD57, CD58, CD79b, CD80, CD86, CD81, CD123, CD133, CD137, CD151, CD171, CD276, CLL1, B7H4, BCMA, VEGFR-2, EGFR, GPC3, PMSA, CEACAM6, c-Met, EGFRVIII, ErbB2/HER2, ErbB3, HER-2, HER3, ErbB4/HER-4, EphA2, IGF1R, GCC, GD2, O-acetyl GD2, O-acetyl GD3, GHRHR, GHR, Flt1, KDR, Flt4, Flt3, CEA, CA125, CTLA-4, GITR, BTLA, TGFBR1, TGFBR2, TGFBR1, IL6R, gp130, Lewis, TNFR1, TNFR2, PD1, PD-L1, PD-L2, PSCA, HVEM, MAGE-A, MSLN, NY-ESO-1, PSMA, RANK, RORI, TNFRSF4, TWEAK-R, LTPR, LIFRP, LRP5, MUCI, MUC16, TCRa, TCRB, TLR7, TLR9, PTCH1, WT-1, Robol, Frizzled, OX40, Notch-I-4, APRIL, CS1, MAGE3, Claudin 18.2, Folate receptor a, Folate receptor B, GPC2, CD70, BAFF-R, and TROP-2.

In some embodiments, the second target is an immune cell specific antigen. In some embodiments, the immune cell antigen can be selected from the group consisting of CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ, and SLAMF7.

In some embodiments, the antibody construct further comprises a Fc fragment. In some embodiments, the Fc fragment is selected from the group consisting of IgG1, IgG2, IgG3 and IgG4. In some embodiments, the Fc fragment further comprises one or more mutations to alter the function of the Fc fragment, for example, to reduce or enhance ADCC (Antibody-dependent cell-mediated cytotoxicity), CDC (Complement dependent cytotoxicity) and/or ADCP (Antibody-dependent cellular phagocytosis) function of the Fc fragment.

### Conjugate

In another aspect, provided herein is a conjugate comprising: (1) the antibody specifically binding to GCC or the antibody construct as described herein, and (2) a drug. In some embodiments, the drug is selected from the group consisting of chemotherapeutic agent, toxin, hormone, enzyme, immunomodulator, chelator, imaging agent, nanoparticle, and radioisotope.

In some embodiments, the drug is a chemotherapeutic agent useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, Astrazeneca), Sutent (SU1 1248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, GlaxoSmithKline), Lonafarnib (SCH 66336), Sorafenib (BAY43-9006, Bayer Labs.), and Gefitinib (IRESSA®, Astrazeneca), AG1478, AG1571 (SU 5271; Sugen), alkylating agents such as Thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins; a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N. J.), ABRAXANE™ Cremophor-free, albumin-engineered nanoparticle formulation of paclitaxel (American Pharmaceutical Partners, Schaumberg, Illinois), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

In some embodiments, the chemotherapeutic agent also includes but is not limited to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; (ii) aromatase inhibitors that inhibit the enzyme aromatase, which regulates estrogen production in the adrenal glands, such as, for example, 4 (5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

In some embodiments, the drug is a cytokine. The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-a, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL).

In some embodiments of the conjugate of the present application, the conjugate further comprises a linker. The terms “linker” as used herein refers to a chemical moiety comprising a covalent bond or a chain of atoms that covalently attaches the antibody as described herein, and/or to the drug as described herein.

### Nucleic Acids, Vectors, Host Cell, and Methods of Producing GCC Antibodies

Nucleic acid molecules encoding the antibody specifically binding to GCC or the antibody construct as described herein are also contemplated. In some embodiments, provided is a nucleic acid encoding the antibody as described herein. In some embodiments, the nucleic acid comprises a polynucleotide sequence having at least 70%, at least 75%, at least 80%, at least 85%, at least 90%, at least 95%, at least 96%, at least 97%, at least 98%, or at least 99% identity with anyone selected from the group consisting of SEQ ID NO. 41-50 and 160-165. In some embodiments, the nucleic acid comprises a polynucleotide sequence of anyone selected from the group consisting of SEQ ID NO. 41-50 and 160-165. The nucleic acid sequences of SEQ ID NOs: 41-50 and 160-165 are provided in the Sequence Summary Table below the Examples.

Also provided are vectors in which a nucleic acid described herein is inserted. In some embodiments, the expression of the antibody specifically binding to GCC as described herein can be achieved by inserting the nucleic acid as described herein into an appropriate expression vector, such that the nucleic acid is operably linked to 5′ and 3′ regulatory elements, including for example a promoter (e.g., a constitutive, regulatable, tissue-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the nucleic acid(s) encoding the antibody specifically binding to GCC as described herein is inducible. In some embodiments, the nucleic acid(s) encoding the antibody specifically binding to GCC as described herein is operably linked to an inducible promoter, including any inducible promoter known in the art. In some embodiments, the nucleic acid(s) encoding the antibody specifically binding to GCC as described herein described herein has been engineered to encode an epitope tag, e.g., to facilitate purification or detection of the polypeptide. Exemplary epitope tags include, but are not limited to, e.g., 6× His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein).

The antibody specifically binding to GCC of the present application can be produced by any means known in the art. Exemplary techniques for polypeptide production are described below; however, these exemplary techniques are provided for illustrative purposes only and are not intended to be limiting.

The antibody specifically binding to GCC of the present application can be produced using recombinant methods. For recombinant production of the antibody specifically binding to GCC of the present application, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the antibody). Many vectors are available. The vector components generally include, but are not limited to, one or more of the following: a signal sequence, an origin of replication, one or more marker genes, an enhancer element, a promoter, and a transcription termination sequence.

Both expression and cloning vectors contain a nucleic acid sequence that enables the vector to replicate in one or more selected host cells, e.g., to allow the vector to replicate independently of the host chromosomal DNA. This sequence can include origins of replication or autonomously replicating sequences. Such sequences are well known for a variety of bacteria, yeast, and viruses. Generally, the origin of replication component is not needed for mammalian expression vectors (the SV40 origin may be used because it contains the early promoter).

Expression and cloning vectors can contain a selection gene or selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Examples of dominant selection use the drugs neomycin, mycophenolic acid and hygromycin. Another example of suitable selectable markers for mammalian cells are those that enable the identification of cells competent to take up the antibody encoding nucleic acid, such as DHFR, glutamine synthetase (GS), thymidine kinase, metallothionein-I and -II, preferably primate metallothionein genes, adenosine deaminase, ornithine decarboxylase, and the like. For example, a Chinese hamster ovary (CHO) cell line deficient in endogenous DHFR activity transformed with the DHFR gene is identified by culturing the transformants in a culture medium containing methotrexate (Mtx), a competitive antagonist of DHFR.

Alternatively, host cells (particularly wild-type hosts that contain endogenous DHFR) transformed or co-transformed with DNA sequences encoding the antibody, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors generally contain a promoter that is recognized by the host organism and is operably linked to nucleic acid encoding the antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoter sequences are known for eukaryotes. Yeast promoters are well known in the art and can include inducible promoters/enhancers regulated by growth conditions. Virtually all eukaryotic genes have an AT-rich region located approximately 25 to 30 bases upstream from the site where transcription is initiated. Examples include without limitation the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. The transcription of the antibody from vectors in mammalian host cells can be controlled, for example, by promoters obtained from the genomes of viruses. The early and late promoters of the SV40 virus are conveniently obtained as an SV40 restriction fragment that also contains the SV40 viral origin of replication. The immediate early promoter of the human cytomegalovirus is conveniently obtained as a HindIII restriction fragment. Alternatively, the Rous Sarcoma Virus long terminal repeat can be used as the promoter.

Transcription of a DNA encoding the antibody as described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus.

Expression vectors used in eukaryotic host cells (yeast, fungi, insect, plant, animal, human, or nucleated cells from other multicellular organisms) will also contain sequences necessary for the termination of transcription and for stabilizing the mRNA.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In addition to prokaryotes, eukaryotic microbes such as filamentous fungi or yeast are suitable cloning or expression hosts for vectors encoding the antibody specifically binding to GCC as described herein. Saccharomyces cerevisiae, or common baker's yeast, is the most commonly used among lower eukaryotic host microorganisms. Certain fungi and yeast strains may be selected in which glycosylation pathways have been “humanized,” resulting in the production of the antibody with a partially or fully human glycosylation pattern. See, e.g., Li et al., Nat. Biotech. 24:210⁻²¹⁵ (2006).

Plant cell cultures of cotton, corn, potato, soybean, petunia, tomato, duckweed (Leninaceae), alfalfa (M. truncatula), and tobacco can also be utilized as hosts.

Suitable host cells for the expression of a glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as NSO and Sp2/0. For a review of certain mammalian host cell lines suitable for production, see, e.g., Yazaki and Wu, Methods in Molecular Biology, Vol. 248 (B. K. C. Lo, ed., Humana Press, Totowa, N.J., 2003), pp. 255-268.

The host cells of the present disclosure may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to one of skill in the art.

When using recombinant techniques, the antibody can be produced intracellularly, in the periplasmic space, or directly secreted into the medium. If the polypeptide is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli.

The polypeptide composition prepared from the cells can be purified using, for example, hydroxyapatite chromatography, hydrophobic interaction chromatography, gel electrophoresis, dialysis, and affinity chromatography, with affinity chromatography being among one of the typically preferred purification steps. In some embodiments, the antibody as described herein comprises an epitope tag (e.g., a tag attached to the antibody specifically binding to GCC as described herein via a cleavable linker) to facilitate purification. Exemplary epitope tags include, but are not limited to, e.g., 6× His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein.

### Chimeric Antigen Receptor and T-Cell Receptor

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as GCC), a transmembrane domain, and an intracellular signaling domain of a T cell and/or other receptors. “CAR-T” refers to a T cell that expresses a CAR.

In another aspect, provided herein is a chimeric antigen receptor (CAR) comprising the antibody construct as described herein. In some embodiments, the CAR comprises the antibody specifically binding to GCC as described herein, a transmembrane domain, and an intracellular signaling domain.

As used herein, the term “T-cell receptor” or “TCR” refers to a molecule on the surface of a T cell or T lymphocyte that is responsible for recognizing an antigen. TCR is a heterodimer which is composed of two different protein chains. In some embodiments, the TCR of the present disclosure consists of an alpha (α) chain and a beta (β) chain and is referred as αβ TCR. αβ TCR recognizes antigenic peptides degraded from protein bound to major histocompatibility complex molecules (MHC) at the cell surface. In some embodiments, the TCR of the present disclosure consists of a gamma (γ) and a delta (δ) chain and is referred as γδ TCR.

In another aspect, provided herein is a T-cell receptor (TCR) comprising the antibody construct as described herein. In some embodiments, the T cell receptor (TCR) comprises the antibody specifically binding to GCC as described herein, wherein the antibody specifically binding to GCC as described herein is linked to: (i) at least one TCR chain selected from an alpha chain, a beta chain, a gamma chain and a delta chain of a T cell receptor; (ii) an epsilon chain, a delta chain, and/or a gamma chain of cluster of differentiation 3 (CD3); or (iii) a CD3 zeta chain.

In some embodiments, also provided herein is a cell expressin the CAR or TCR as described herein. In some embodiments, the cell is an immunce cell. In some embodiments, the cell can be selected from the group consisting of a T cell, an NK cell, a peripheral blood mononuclear cell (PBMC), a hematopoietic stem cell, a pluripotent stem cell, or an embryonic stem cell.

### Pharmaceutical Composition

In another aspect, provided herein is a pharmaceutical composition comprising the the antibody, the nucleic acid, the conjugate and/or the cell as described herein, and a pharmaceutically acceptable excipient.

Any suitable excipient can be used in the pharmaceutical composition of the present application. In some embodiments, the exicipient includes but is not limited to diluents; carriers; excipients; stabilizers; buffers such as phosphate, citrate, and other organic acids buffers; antioxidants including ascorbic acid and methionine; preservatives such as octadecyldimethylbenzyl ammonium chloride, hexamethonium chloride, enzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Methods

Also provided herein is a method of treating a disorder in a subject in need thereof, comprising administrating an effective amount of the pharmaceutical composition as described herein to the subject. In some embodiments, the method comprising administrating the antibody as described herein, the nucleic acid as described herein, the vector as described herein, the cell as described herein, and/or the pharmaceutical composition as described herein to the subject. Also provided herein is a method for treating a disorder associated with the undesired presence of GCC+ cells, comprising administrating the antibody as described herein, the nucleic acid as described herein, the vector as described herein, the cell as described herein, and/or the pharmaceutical composition as described herein to the subject.

In some embodiments, the disorder is a proliferative disorder. In some embodiments, the disorder is cancer.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Generation of Single Domain Antibodies (sdAb) Specifically Binding to GCC

### 1.1 Animal Immunization and Phage Library Construction

Two alpacas were subcutaneously injected human GCC×10*His every 1-2 weeks for 6 doses, 100 to 200 μg antigen per dose. Blood samples were collected at different time points, and induction of an antigen specific humoral immune response was verified by ELISA using the serum prepared from the blood samples.

7 days after the final immunization, 50 mL blood sample was collected from each animal and approximately 1×10⁸ peripheral blood mononuclear cells (PBMCs) were obtained from the blood sample by density gradient centrifugation on Ficoll-Paque PLUS (GE Healthcare, Little Chalfont, UK). Total RNA was extracted from the PBMCs and transcribed into cDNA using an oligo-dT primer and SuperScript III First-Strand Synthesis SuperMix System (Invitrogen, Carlsbad, CA, USA) according to the manufacturers' recommendations.

The purified cDNA was then used as template to amplify the repertoire of Ig heavy chain-encoding gene segments by using signal peptide domain specific primers and CH2 domain specific primers. Fragments of approximately 700 bp (representing heavy-chain IgGs that lack a CH1 domain) were isolated from agarose gel and purified by QIAquick Gel Extraction Kit (Qiagen, Hilden, Germany). The purified fragments were further used as templates to amplify the VHH repertoire by using framework1 (FR1) and framework4 (FR4) specific primer pairs.

After amplification, purified VHH genes were cloned into phagemid vector pFL249, and electrotransformed into E. coli. TG1. After transformation, the TG1 cells were cultured in SOC medium at 37° C. for 1 hour, then inoculated onto plates containing solid 2YT medium supplemented with 100 μg/mL Carb and 1% (w/v) glucose and cultured at 37° C. overnight. The next day, the colonies were scraped into liquid 2YT medium supplemented with 1/3 (v/v) of 80% glycerol and stored at −80° C. The library size was about 1×10⁸, VHH insert ratio was above 95%, and phage display ratio is above 85%.

### 1.2 Antibody Panning and Screening

The constructed library was subject to four rounds of panning, followed by screening of binding capability via ELISA. 30 screened clones were further ranked by SPR and 10 clones were selected for sequencing. The sequences is as shown in Table 3.

TABLE 3
Sequences of exemplary anti-GCC single domain antibodies

| No. | Sequence |
|---|---|
| 1 | QVQLVESGGGLVQAGGSLRLSCTGSARPFSNYGMGWFRQAPGKEREFVAAISGGGVYTDF |
| | ADAVKGRFTISRDNAKNTVYLQMNSLKPEDTAVYYCARARTARVVSRRHEYDYWGQGTQV |
| | TVSS |
| 2 | QVQLVESGGGLVQAGGSLRLSCTASGGTFVNYGMGWFRQAPGKEREFVAAISGGGVYRDF |
| | ADSVKGRFTISRDNAKNTVYLQMNSLKPEDTALYYCARARTPRVVNRRHEYDYWGQGTQV |
| | TVSS |
| 3 | QVQFVESGGGLVQAGGSLRLSCTASGGIFSNYGMGWFRQAPGKEREFVAAISGGGVYTDF |
| | ADSVKGRFTISRDRAKNTVYLQMNSLKPEDTAVYYCARARTARLTSRRHDYDYWGQGTQV |
| | TVSS |
| 4 | QSQLVESGGGLVQPGGSLRLSCAASGSTLDHYATAWFRRAPGKEREGISYISSGGGSTYY |
| | VDSVKGRFTISRDNAKRTVYLQMNSLKPEDTAVYFCASKRRENTGWSEPSDYNVWGQGTQ |
| | VTVTS |
| 5 | AVQLVESGGGLVQPGGSLRLSCAASGSTISINVMGWYRQAPGKQRDLVAAISSVGSTYYA |
| | DSVKGRFTISRDNAKNTVYLQMNSLKPEDTAVYYCNAKSLISDYWGQGTQVTVSS |
| 6 | QVQVVESGGGLVQAGGSLRLSCAASGRAFSSYAMGWFRQAPGKQRELVAVITSGGSTNYA |
| | DSVKGRFTISRDNAKNTVYLQMNSLKPEDTAVYYCAAGTGYGGGFLIGYDYWGQGTQVTV |
| | SS |
| 7 | QVQLVESGGGLVQPGGSLRLSCAASGSRFMTSSMGWYRQTPGKQREWVATATSSDSTLYA |
| | DAVKGRFTISRDNAKNTVYLQMNSLKPQDTAVYYCFTTPPTFWGHGTQVTVSS |
| 8 | EVDLVESGGGLVQPGGSLRLSCGASGFTFSHYAMSWVRQAPGKALEWVSDISSGGGITKY |
| | ADSVKGRFTISRDDAQNTLYLQMNSLKPEDTAVYYCAKASAIGYLAYWMRNPVEYDTRGQ |
| | GTLVTVSS |
| 9 | QKQLVESGGGLVQPGGSLRLSCAASGETLSNYNIGWFRQAPGKEREGVSCISQIGFTNYA |
| | DSVKGRFTLFRASAENTVYLQMNSLKPEDTAVYYCAVTGGRWASCSFLPLRQDFESWGQG |
| | TLVTVSS |
| 10 | AVQLVESGGGMVQPGGSLRLSCAASGFTFGAYDMTWVRQAPGKGAEWVSAINSGGDRTYY |
| | TDSVKGRFTISRDNAKNTLYLQMNSLKPADTAKYYCGTKIPQFLRGQGTQVTVSS |
| 118 | QPQVVESGGGLVQPGRSLTLSCAAYGTISKPYAMGWYRQAPEKQREWVATIFSDHSTNYA |
| | GAVKGRFTISRDNAKNTVYLQMNSLKPEDTAVYYCLPLGATFAGVAWGQGTQVTVSS |
| 119 | EVDLVESGGGLVQPGGSLRLSCAASQSIFSINVISWYRQAPGKQRELVAAIHSGGSTNYA |
| | DSVKGRFTISRDGTKNTVYLQMNSLKPEDTAVYYCHADTDFDRTFGSWGQGTQVTVSS |
| 120 | QVQLVESGGGLVQPGGSLRLSCAASGFTFGTYDMVWVRQAPGKGAEWVSGINSGGGRTYY |
| | ADSVKGRFTISRDNAKNTLYLQMNSLKSEDTAVYYCGTGDGRSRTYTGQGTQVTVSS |
| 121 | EVOLVESGGGLVQAGDSLRLSCAASGGTFSNYAMGWFRQAPGKDREFVSIVSQIGGVTRY |
| | ADSVKGRFTISRDNAKNMLYLQMNSLKPEDTAVYYCAAVRTNSRFYSDFEDSIVSWGQGT |
| | QVTVSS |
| 122 | EVOLVESGGGLVQPGGSLRLSCAASGSRFMTNAMGWWRQAPGKQREWVATATSNSTLYAE |
| | AVKGRFTISRDNAKNTVYLQMNSLKPQDTGVYYCFTTPPTAWGHGTQVTVSS |
| 123 | QVQLVESGGGVVQPGGSLRLSCAASGFTFGSYDMTWVRQAPGKGAEWVAAIDRGGTSTYY |
| | ADSVKGRFTISRDNAKNTLYLQMNSLKPEDTAVYYCGTREDGRATTGQGTQVTVSS |

### Example 2. Characterization of the Exemplary Anti-GCC Single Domain Antibodies of the Present Application

2.1 Binding Affinity of Anti-GCC sdAbs Via Surface Plasmon Resonance (SPR)

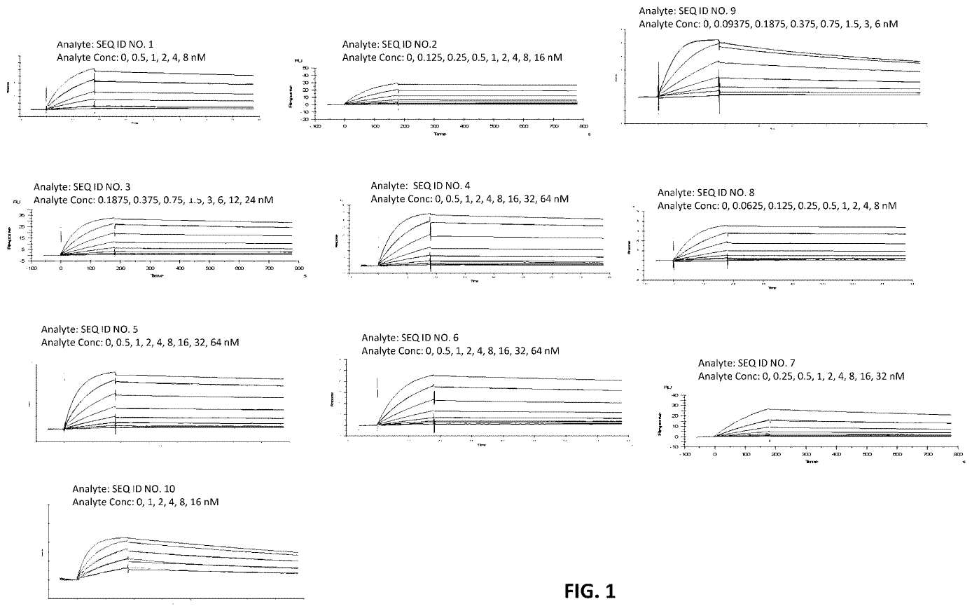

Anti-GCC sdAbs were diluted with 1×HBS-EP buffer. Human GUCY2C was captured on anti-human Fc capturing Ab-immobilized sCM5 chip. Then anti-GCC sdAb samples were injected with a flow rate of 10 μl/min for 180 sec, followed by dissociation for 800 sec. The binding curves were locally fitted with Biacore® T200 analysis software (BIAevaluation) using a 1:1 Langmuir binding model. The result is as shown in Table 4 and FIG. 1.

TABLE 4
Binding affinities of the exemplary anti-
GCC sdAbs of the present application

| ID | Kₐ (1/Ms) | K_{d} (1/s) | K_{D} (M) |
|---|---|---|---|
| SEQ ID NO. 1 | 3.57E+05 | 2.00E−04 | 5.604E−10 |
| SEQ ID NO. 2 | 6.15E+05 | 4.67E−05 | 7.594E−11 |
| SEQ ID NO. 3 | 8.27E+05 | 1.80E−04 | 2.177E−10 |
| SEQ ID NO. 4 | 3.59E+05 | 1.49E−04 | 4.14E−10 |
| SEQ ID NO. 5 | 3.30E+05 | 1.42E−04 | 4.31E−10 |
| SEQ ID NO. 6 | 2.28E+05 | 1.72E−04 | 7.53E−10 |
| SEQ ID NO. 7 | 1.56E+05 | 3.95E−04 | 2.53E−09 |
| SEQ ID NO. 8 | 2.59E+06 | 6.70E−05 | 2.59E−11 |
| SEQ ID NO. 9 | 8.84E+06 | 1.03E−03 | 1.16E−10 |
| SEQ ID NO. 10 | 1.64E+06 | 7.02E−04 | 4.27E−10 |

2.2 Binding Affinities of the Exemplary Anti-GCC sdAbs of the Present Application with T84 Cells

T84 cells expressing GCC were used to evaluate the binding affinity of the anti-GCC sdAbs. Briefly, 50 μL T84 cells at density of 3×10⁶ cells/mL were incubated with 50 μL anti-GCC sdAbs at 4° C. for 2 hours. After incubation, the cells were washed three times with 400 μL FACS buffer, resuspended with PE-anti human Fc 2nd antibody (eBioscience 12-4998-82, 1:1000 dilution) and incubated at 4° C. for 60 min. Then the cells were collected and subject to FACS analysis. The result is as shown in Table 5A and 5B below.

TABLE 5A
Geometric mean MFI of the exemplary anti-GCC
sdAbs of the present application (300 nm)

| ID | MFI |
|---|---|
| Human IgG1 isotype | 30.7 |
| Blank | 30.7 |
| SEQ ID NO. 1 | 790 |
| SEQ ID NO. 2 | 721 |
| SEQ ID NO. 3 | 910 |
| SEQ ID NO. 4 | 949 |
| SEQ ID NO. 5 | 371 |
| SEQ ID NO. 6 | 298 |
| SEQ ID NO. 7 | 268 |
| SEQ ID NO. 8 | 669 |
| SEQ ID NO. 9 | 447 |

TABLE 5B
Geometric mean MFI of the anti-GCC sdAbs
of the present application (300 nm)

| ID | MFI |
|---|---|
| Human IgG1 isotype | 17.7 |
| Blank | 15.0 |
| SEQ ID NO. 10 | 265 |

### Example 3. Internalization of the Anti-GCC Single Domain Antibodies of the Present Application

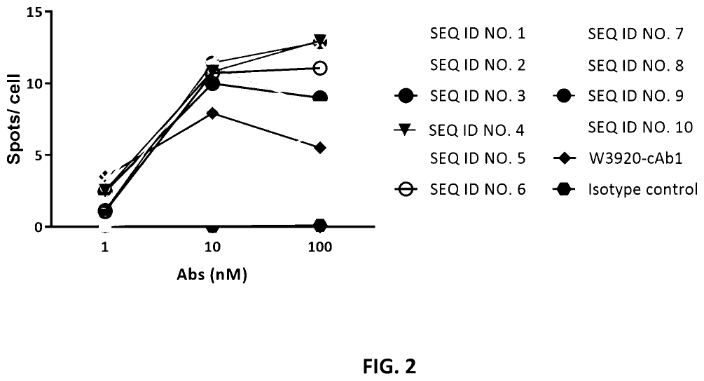

Anti-His antibody was labeled with pHrodo iFL Red STP ester amine reactive dye and purified by Zeba™ spin desalting column according to the manufacturer's instructions. GCC overexpressing 293F cells expressing GCC were seeded on 96-well plates at a density of 1.5×10⁴ cells/well in complete culture medium containing 10% FBS, and cultured at 37° C., 5% CO₂ overnight. Anti-GCCs of the present application at different concentrations were mixed with the pHrodo iFL Red/anti-His antibody conjugates at a ratio of 1:2 (unit: mol/L). The GCC overexpressing 293F cells were then incubated with the mixture at 4° C. for 6 hours. After incubation, the cells were treated with 100 μL Hoechst 33342 diluted in DPBS (4 μg/ml) at 4° C. for 15 min. The fluorescence intensity of GCC overexpressing 293F cells was analyzed by Perkin Elmer Operetta CLS High-Content Analysis System. The internalization result is as shown in FIG. 2.

### Example 4. Antibody Binning

Anti-GCC sdAbs were diluted with 1×HBS-EP buffer. Human GUCY2C was captured on anti-human Fc capturing Ab-immobilized sCM5 chip. Then anti-GCC sdAb samples were injected with a flow rate of 30 μl/min for 120 sec, followed by dissociation for 120 sec. Then the competitive anti-GCC sdAb samples were injected with a flow rate of 30 μl/min for 120 sec, followed by dissociation for 120 sec. The result was locally fitted with Biacore® T200 analysis software (BIAevaluation), as shown in Table 6. Based on Table 6, it can be seen that the anti-GCC sdAbs with SEQ ID NO. 1-4 substantially bind to the same epitope in GCC antigen; the anti-GCC sdAbs with SEQ ID NO. 5 and 6 substantially bind to the same epitope in GCC antigen; and the anti-GCC sdAbs with SEQ ID NO. 7-9 substantially bind to the same epitope in GCC antigen.

TABLE 6
Antibody binning

| SEQ ID NO. | 1 | 2 | 3 | 4 | 5 | 6 | 8 | 9 | 7 |
|---|---|---|---|---|---|---|---|---|---|
| 1 | Y | Y | Y | Y | N | N | N | N | N |
| 2 | Y | Y | Y | Y | N | N | N | N | N |
| 3 | Y | Y | Y | Y | N | N | N | N | N |
| 4 | Y | Y | Y | Y | N | N | N | N | N |
| 5 | N | N | N | N | Y | Y | N | N | N |
| 6 | N | N | N | N | Y | Y | N | N | N |
| 8 | N | N | N | N | N | N | Y | Y | N |
| 9 | N | N | N | N | N | N | Y | Y | Y |
| 7 | N | N | N | N | N | N | N | Y | Y |

### Example 5. Humanization of Exemplary Anti-GCC Single Domain Antibodies of the Present Application

Selected anti-GCC sdAbs were further subject to humanization. Briefly, the amino acid sequences of the hits in Table 3 were compared against the available database of human Ig gene sequences to obtain the best-matching human germline Ig gene sequences. Then coding sequences of CDR1, CDR2 and CDR3 of each selected sdAbs were grafted onto the framework of its best-matching human germline Ig gene, respectively, to obtain the humanized sdAbs as shown in Table 7 below.

TABLE 7
Humanized anti-GCC sdAbs

| No. | Sequence |
|---|---|
| 82 | EVQLVESGGGLVQPGGSLRLSCAASARPFSNYGMSWVRQA |
| | PGKGLEWVSAISGGGVYTYYADSVKGRFTISRDNSKNTLY |
| | LQMNSLRAEDTAVYYCARARTARVVSRRHEYDYWGQGTQV |
| | TVSS |
| 83 | EVQLVESGGGLVQPGGSLRLSCAASARPFSNYGMGWFRQA |
| | PGKEREFVAAISGGGVYTYYADSVKGRFTISRDNSKNTLY |
| | LQMNSLRAEDTAVYYCARARTARVVSRRHEYDYWGQGTQV |
| | TVSS |
| 84 | EVQLVESGGGLVQPGGSLRLSCTGSARPFSNYGMGWFRQA |
| | PGKEREFVAAISGGGVYTDFADSVKGRFTISRDNSKNTLY |
| | LQMNSLRAEDTAVYYCARARTARVVSRRHEYDYWGQGTQV |
| | TVSS |
| 85 | EVQLVESGGGLVQPGGSLRLSCAASARPFSNYGMGWFRQA |
| | PGKEREFVAAISGGGVYTYYADSVKGRFTISRDNSRNTLY |
| | LQMNSLRAEDTAVYYCARARTARVVSRRHEYDYWGQGTQV |
| | TVSS |
| 86 | EVQLVESGGGLVQPGGSLRLSCTGSARPFSNYGMGWFRQA |
| | PGNEREFVAAISGGGVYTDFADSVKGRFTISRDNSKNTLY |
| | LQMNSLRAEDTAVYYCARARTARVVSRRHEYDYWGQGTQV |
| | TVSS |
| 87 | EVQLVESGGGLVQPGGSLRLSCAASGGTFVNYGMSWVRQA |
| | PGKGLEWVSAISGGGVYRYYADSVKGRFTISRDNSKNTLY |
| | LQMNSLRAEDTAVYYCARARTPRVVNRRHEYDYWGQGTQV |
| | TVSS |
| 88 | EVQLVESGGGLVQPGGSLRLSCAASGGTFVNYGMGWFRQA |
| | PGKEREFVAAISGGGVYRYYADSVKGRFTISRDNSKNTLY |
| | LQMNSLRAEDTAVYYCARARTPRVVNRRHEYDYWGQGTQV |
| | TVSS |
| 89 | EVQLVESGGGLVQPGGSLRLSCTASGGTFVNYGMGWFRQA |
| | PGKEREFVAAISGGGVYRDFADSVKGRFTISRDNSKNTLY |
| | LQMNSLRAEDTAVYYCARARTPRVVNRRHEYDYWGQGTQV |
| | TVSS |
| 90 | EVQLVESGGGLVQPGGSLRLSCAASGGTFVNYGMGWFRQA |
| | PGKEREFVAAISGGGVYRYYADSVKGRFTISRDNSRNTLY |
| | LQMNSLRAEDTAVYYCARARTPRVVNRRHEYDYWGQGTQV |
| | TVSS |
| 91 | EVQLVESGGGLVQPGGSLRLSCAASGGIFSNYGMSWVRQA |
| | PGKGLEWVSAISGGGVYTYYADSVKGRFTISRDNSKNTLY |
| | LQMNSLRAEDTAVYYCARARTARLTSRRHDYDYWGQGTQV |
| | TVSS |
| 92 | EVQLVESGGGLVQPGGSLRLSCAASGGIFSNYGMGWFRQA |
| | PGKEREFVAAISGGGVYTYYADSVKGRFTISRDNSKNTLY |
| | LQMNSLRAEDTAVYYCARARTARLTSRRHDYDYWGQGTQV |
| | TVSS |
| 93 | EVQLVESGGGLVQPGGSLRLSCTASGGIFSNYGMGWFRQA |
| | PGKEREFVAAISGGGVYTDFADSVKGRFTISRDNSKNTLY |
| | LQMNSLRAEDTAVYYCARARTARLTSRRHDYDYWGQGTQV |
| | TVSS |
| 94 | EVQLVESGGGLVQPGGSLRLSCAASGGIFSNYGMGWFRQA |
| | PGKEREFVAAISGGGVYTYYADSVKGRFTISRDNSRNTLY |
| | LQMNSLRAEDTAVYYCARARTARLTSRRHDYDYWGQGTQV |
| | TVSS |
| 95 | QSQLVESGGGLVQPGGSLRLSCAASGSTLDHYAMSWVRQA |
| | PGKGLEWVSAISSGGGSTYYADSVKGRFTISRDNSKNTLY |
| | LQMNSLRAEDTAVYYCASKRRENTGWSEPSDYNVWGQGTQ |
| | VTVTS |
| 96 | QSQLVESGGGLVQPGGSLRLSCAASGSTLDHYATAWFRRA |
| | PGKEREGISYISSGGGSTYYADSVKGRFTISRDNSKNTLY |
| | LQMNSLRAEDTAVYYCASKRRFNTGWSEPSDYNVWGQGTQ |
| | VTVTS |
| 97 | QSQLVESGGGLVQPGGSLRLSCAASGSTLDHYATAWFRRA |
| | PGKEREGISYISSGGGSTYYVDSVKGRFTISRDNSKNTLY |
| | LQMNSLRAEDTAVYFCASKRRFNTGWSEPSDYNVWGQGTQ |
| | VTVTS |
| 98 | QSQLVESGGGLVQPGGSLRLSCAASGSTLDHYATAWFRRA |
| | PGKEREGISYISSGGGSTYYADSVAGRFTISRDNSRNTLY |
| | LQMNSLRAEDTAVYYCASKRRENTGWSEPSDYNVWGQGTQ |
| | VTVTS |
| 99 | AVQLVESGGGLVQPGGSLRLSCAASGSTISINVMSWVRQA |
| | PGKGLEWVSVISSVGSTYYADSVKGRFTISRDNSKNTLYL |
| | QMNSLRAEDTAVYYCNAKSLISDYWGQGTQVTVSS |
| 100 | AVQLVESGGGLVQPGGSLRLSCAASGSTISINVMGWYRQA |
| | PGKQRDLVAAISSVGSTYYADSVKGRFTISRDNSKNTLYL |
| | QMNSLRAEDTAVYYCNAKSLISDYWGQGTQVTVSS |
| 101 | AVQLVESGGGLVQPGGSLRLSCAASGSTISINVMGWYRQA |
| | PGKQRDLVAAISSVGSTYYADSVAGRFTISRDNSRNTLYL |
| | QMNSLRAEDTAVYYCNAKSLISDYWGQGTQVTVSS |
| 102 | EVQLVESGGGLVQPGGSLRLSCAASGRAFSSYAMSWVRQA |
| | PGKGLEWVSAITSGGSTYYADSVKGRFTISRDNSKNTLYL |
| | QMNSLRAEDTAVYYCAAGTGYGGGFLIGYDYWGQGTQVTV |
| | SS |
| 103 | EVQLVESGGGLVQPGGSLRLSCAASGRAFSSYAMGWFRQA |
| | PGKQRELVAVITSGGSTYYADSVKGRFTISRDNSKNTLYL |
| | QMNSLRAEDTAVYYCAAGTGYGGGFLIGYDYWGQGTQVTV |
| | SS |
| 104 | EVQLVESGGGLVQPGGSLRLSCAASGRAFSSYAMGWFRQA |
| | PGKQRELVAVITSGGSTNYADSVKGRFTISRDNSKNTLYL |
| | QMNSLRAEDTAVYYCAAGTGYGGGFLIGYDYWGQGTQVTV |
| | SS |
| 105 | EVQLVESGGGLVQPGGSLRLSCAASGRAFSSYAMGWFRQA |
| | PGKQRELVAVITSGGSTYYADSVKGRFTISRDNSRNTLYL |
| | QMNSLRAEDTAVYYCAAGTGYGGGFLIGYDYWGQGTQVTV |
| | SS |
| 106 | EVQLVESGGGLVQPGGSLRLSCAASGSRFMTSSMNWVRQA |
| | PGKGLEWVSTATSSDSTLYADSVKGRFTISRDNAKNSLYL |
| | QMNSLRAEDTAVYYCFTTPPTFWGHGTQVTVSS |
| 107 | EVQLVESGGGLVQPGGSLRLSCAASGSRFMTSSMGWYRQT |
| | PGKQREWVATATSSDSTLYADSVKGRFTISRDNAKNSLYL |
| | QMNSLRAEDTAVYYCFTTPPTFWGHGTQVTVSS |
| 108 | EVQLVESGGGLVQPGGSLRLSCAASGSRFMTSSMGWYRQT |
| | PGKQREWVATATSSDSTLYADSVKGRFTISRDNARNSLYL |
| | QMNSLRAEDTAVYYCFTTPPTFWGHGTQVTVSS |
| 109 | EVQLVESGGGLVQPGGSLRLSCAASGSRFMTSSMGWYRQT |
| | PGNQREWVATATSSDSTLYADSVKGRFTISRDNAKNSLYL |
| | QMNSLRAEDTAVYYCFTTPPTFWGHGTQVTVSS |
| 110 | EVQLVESGGGLVQPGGSLRLSCAASGSRFMTSSMGWYRQT |
| | PGNGREWVATATSSDSTLYADSVKGRFTISRDNAKNSLYL |
| | QMNSLRAEDTAVYYCFTTPPTFWGHGTQVTVSS |
| 111 | EVQLVESGGGLVQPGGSLRLSCAASGFTFSHYAMSWVRQA |
| | PGKGLEWVSAISSGGGITYYADSVKGRFTISRDNSKNTLY |
| | LQMNSLRAEDTAVYYCAKASAIGYLAYWMRNPVEYDTRGQ |
| | GTLVTVSS |
| 112 | EVQLVESGGGLVQPGGSLRLSCAASGFTFSHYAMSWVRQA |
| | PGKALEWVSDISSGGGITYYADSVKGRFTISRDNSKNTLY |
| | LQMNSLRAEDTAVYYCAKASAIGYLAYWMRNPVEYDTRGQ |
| | GTLVTVSS |
| 113 | EVQLVESGGGLVQPGGSLRLSCGASGFTFSHYAMSWVRQA |
| | PGKALEWVSDISSGGGITKYADSVKGRFTISRDNSKNTLY |
| | LQMNSLRAEDTAVYYCAKASAIGYLAYWMRNPVEYDTRGQ |
| | GTLVTVSS |
| 114 | QVQLVESGGGLVQPGGSLRLSCAASGFTFSHYAMSWVRQA |
| | PGKALEWVSDISSGGGITYYADSVAGRFTISRDNSRNTLY |
| | LQMNSLRAEDTAVYYCAKASAIGYLAYWMRNPVEYDTRGQ |
| | GTLVTVSS |
| 115 | QVQLVESGGGLVKPGGSLRLSCAASGETLSNYNMSWIRQA |
| | PGKGLEWVSYISQIGFTNYADSVKGRFTISRDNAKNSLYL |
| | QMNSLRAEDTAVYYCAVTGGRWASCSFLPLRQDFESWGQG |
| | TLVTVSS |
| 116 | QVQLVESGGGLVKPGGSLRLSCAASGETLSNYNIGWFRQA |
| | PGKEREGVSCISQIGFTNYADSVKGRFTISRDNAKNSLYL |
| | QMNSLRAEDTAVYYCAVTGGRWASCSFLPLRQDFESWGQG |
| | TLVTVSS |
| 117 | QVQLVESGGGLVQPGGSLRLSCAASGETLSNYNIGWFRQA |
| | PGKEREGVSCISQIGFTNYADSVKGRFTISRDNARNSLYL |
| | QMNSLRAEDTAVYYCAVTGGRWASCSFLPLRQDFESWGQG |
| | TLVTVSS |

### Example 6. Characterization of Humanized Anti-GCC Single Domain Antibodies of the Present Application

Exemplary humanized anti-GCC sdAbs of the present application were diluted with 1×HBS-EP buffer. Human GUCY2C was captured on anti-human Fc capturing Ab-immobilized sCM5 chip. Then the humanized anti-GCC sdAb samples were injected with a flow rate of 10 μl/min for 180 sec, followed by dissociation for 800 sec. The binding curves were locally fitted with Biacore® T200 analysis software (BIAevaluation) using a 1:1 Langmuir binding model. The result is as shown in Table 8.

TABLE 8
Binding affinities of exemplary humanized
anti-GCC sdAbs of the present application

| ID | Kₐ (1/Ms) | K_{d} (1/s) | K_{D} (M) |
|---|---|---|---|
| SEQ ID NO. 83 | 1.53E+06 | 1.77E−04 | 1.15E−10 |
| SEQ ID NO. 85 | 1.66E+06 | 3.09E−04 | 1.87E−10 |
| SEQ ID NO. 86 | 7.68E+05 | 1.81E−04 | 2.35E−10 |
| SEQ ID NO. 107 | 2.61E+05 | 5.31E−04 | 2.03E−09 |
| SEQ ID NO. 108 | 2.31E+05 | 5.46E−04 | 2.37E−09 |
| SEQ ID NO. 109 | 1.81E+05 | 2.13E−03 | 1.18E−08 |
| SEQ ID NO. 110 | 1.83E+05 | 1.00E−02 | 5.46E−08 |
| SEQ ID NO. 88 | 2.17E+06 | 3.37E−04 | 1.55E−10 |
| SEQ ID NO. 116 | 4.14E+06 | 6.36E−02 | 1.54E−08 |
| SEQ ID NO. 92 | 1.04E+06 | 7.88E−04 | 7.59E−10 |
| SEQ ID NO. 94 | 1.49E+06 | 6.33E−04 | 4.25E−10 |
| SEQ ID NO. 96 | 8.26E+04 | 1.43E−04 | 1.74E−09 |
| SEQ ID NO. 97 | 6.13E+04 | 2.26E−04 | 3.68E−09 |
| SEQ ID NO. 112 | 3.91E+04 | 4.62E−03 | 1.18E−07 |
| SEQ ID NO. 113 | 2.15E+06 | 2.77E−03 | 1.29E−09 |
| SEQ ID NO. 114 | 2.26E+04 | 4.40E−03 | 1.95E−07 |
| SEQ ID NO. 100 | 5.91E+05 | 1.91E−04 | 3.24E−10 |
| SEQ ID NO. 101 | 3.80E+05 | 2.18E−04 | 5.74E−10 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

SEQUENCE LISTING

| No. | Sequence |
|---|---|
| 1 | QVQLVESGGGLVQAGGSLRLSCTGSARPFSNYGMGWFRQAPGKEREFVAAISGGGVYTDF |
| | ADAVKGRFTISRDNAKNTVYLQMNSLKPEDTAVYYCARARTARVVSRRHEYDYWGQGTQV |
| | TVSS |
| 2 | QVQLVESGGGLVQAGGSLRLSCTASGGTFVNYGMGWFRQAPGKEREFVAAISGGGVYRDF |
| | ADSVKGRFTISRDNAKNTVYLQMNSLKPEDTALYYCARARTPRVVNRRHEYDYWGQGTQV |
| | TVSS |
| 3 | QVQFVESGGGLVQAGGSLRLSCTASGGIFSNYGMGWFRQAPGKEREFVAAISGGGVYTDF |
| | ADSVKGRFTISRDRAKNTVYLQMNSLKPEDTAVYYCARARTARLTSRRHDYDYWGQGTQV |
| | TVSS |
| 4 | QSQLVESGGGLVQPGGSLRLSCAASGSTLDHYATAWFRRAPGKEREGISYISSGGGSTYY |
| | VDSVKGRFTISRDNAKRTVYLQMNSLKPEDTAVYFCASKRRENTGWSEPSDYNVWGQGTQ |
| | VTVTS |
| 5 | AVQLVESGGGLVQPGGSLRLSCAASGSTISINVMGWYRQAPGKQRDLVAAISSVGSTYYA |
| | DSVKGRFTISRDNAKNTVYLQMNSLKPEDTAVYYCNAKSLISDYWGQGTQVTVSS |
| 6 | QVQVVESGGGLVQAGGSLRLSCAASGRAFSSYAMGWFRQAPGKQRELVAVITSGGSTNYA |
| | DSVKGRFTISRDNAKNTVYLQMNSLKPEDTAVYYCAAGTGYGGGFLIGYDYWGQGTQVTV |
| | SS |
| 7 | QVQLVESGGGLVQPGGSLRLSCAASGSRFMTSSMGWYRQTPGKQREWVATATSSDSTLYA |
| | DAVKGRFTISRDNAKNTVYLQMNSLKPQDTAVYYCFTTPPTFWGHGTQVTVSS |
| 8 | EVDLVESGGGLVQPGGSLRLSCGASGFTFSHYAMSWVRQAPGKALEWVSDISSGGGITKY |
| | ADSVKGRFTISRDDAQNTLYLQMNSLKPEDTAVYYCAKASAIGYLAYWMRNPVEYDTRGQ |
| | GTLVTVSS |
| 9 | QKQLVESGGGLVQPGGSLRLSCAASGETLSNYNIGWFRQAPGKEREGVSCISQIGFTNYA |
| | DSVKGRFTLFRASAENTVYLQMNSLKPEDTAVYYCAVTGGRWASCSFLPLRQDFESWGQG |
| | TLVTVSS |
| 10 | AVQLVESGGGMVQPGGSLRLSCAASGFTFGAYDMTWVRQAPGKGAEWVSAINSGGDRTYY |
| | TDSVKGRFTISRDNAKNTLYLQMNSLKPADTAKYYCGTKIPQFLRGQGTQVTVSS |
| 11 | ARPFSNYG |
| 12 | GGTFVNYG |
| 13 | GGIFSNYG |
| 14 | GSTLDHYA |
| 15 | GSTISINV |
| 16 | GRAFSSYA |
| 17 | GSRFMTSS |
| 18 | GFTFSHYA |
| 19 | GETLSNYN |
| 20 | GFTFGAYD |
| 21 | ISGGGVYT |
| 22 | ISGGGVYR |
| 23 | ISGGGVYT |
| 24 | ISSGGGST |
| 25 | ISSVGST |
| 26 | ITSGGST |
| 27 | ATSSDST |
| 28 | ISSGGGIT |
| 29 | ISQIGFT |
| 30 | INSGGDRT |
| 31 | ARARTARVVSRRHEYDY |
| 32 | ARARTPRVVNRRHEYDY |
| 33 | ARARTARLTSRRHDYDY |
| 34 | ASKRRFNTGWSEPSDYNV |
| 35 | NAKSLISDY |
| 36 | AAGTGYGGGFLIGYDY |
| 37 | FTTPPTF |
| 38 | AKASAIGYLAYWMRNPVEYDT |
| 39 | AVTGGRWASCSFLPLRQDFES |
| 40 | GTKIPQFL |
| 41 | CAGGTGCAGCTCGTGGAATCTGGGGGAGGATTGGTGCAGGCCGGGGGGTCTCTGAGACTC |
| | TCCTGTACAGGCTCTGCACGCCCCTTCAGTAACTATGGCATGGGCTGGTTCCGCCAGGCT |
| | CCAGGGAAGGAGCGTGAGTTTGTTGCAGCTATTAGCGGTGGTGGTGTCTACACAGACTTC |
| | GCAGACGCCGTGAAGGGCCGATTCACCATCTCCAGAGACAATGCCAAGAACACGGTGTAT |
| | CTGCAAATGAACAGCCTGAAACCTGAGGACACGGCCGTTTATTACTGTGCACGCGCGCGT |
| | ACAGCGCGCGTTGTTAGTAGACGTCATGAGTATGACTACTGGGGCCAGGGGACCCAGGTC |
| | ACCGTCTCCTCA |
| 42 | CAGGTGCAGCTTGTGGAGTCTGGGGGAGGATTGGTGCAGGCCGGGGGGTCTCTGAGACTC |
| | TCCTGTACAGCCTCTGGAGGCACCTTTGTTAACTATGGCATGGGCTGGTTCCGCCAGGCT |
| | CCAGGGAAGGAGCGTGAGTTTGTTGCAGCTATTAGCGGTGGTGGTGTCTACCGAGACTTC |
| | GCAGACTCCGTGAAGGGCCGATTCACCATCTCCAGAGACAATGCCAAGAACACGGTGTAT |
| | CTGCAAATGAACAGCCTGAAACCTGAGGACACGGCCCTTTATTACTGTGCACGCGCGCGT |
| | ACACCTCGTGTTGTTAATAGACGTCATGAGTATGACTACTGGGGCCAGGGGACCCAGGTC |
| | ACCGTCTCCTCA |
| 43 | CAGGTGCAGTTCGTGGAATCTGGGGGAGGATTGGTGCAGGCCGGGGGGTCTCTGAGACTC |
| | TCCTGTACAGCCTCTGGAGGCATCTTCAGTAACTATGGCATGGGCTGGTTCCGCCAGGCT |
| | CCAGGGAAGGAGCGTGAGTTTGTTGCAGCTATTAGCGGTGGTGGTGTCTACACAGACTTC |
| | GCAGACTCCGTGAAGGGCCGATTCACCATCTCCAGAGACCGTGCCAAGAACACGGTGTAT |
| | CTGCAAATGAACAGCCTGAAACCTGAGGACACGGCCGTTTATTACTGTGCACGCGCGCGT |
| | ACAGCACGGCTTACTAGTAGACGTCATGACTATGACTACTGGGGCCAGGGGACCCAGGTC |
| | ACCGTCTCCTCA |
| 44 | CAGTCGCAGCTCGTGGAATCTGGGGGAGGCTTGGTGCAGCCTGGGGGGTCTCTGAGACTC |
| | TCCTGTGCAGCCTCTGGATCCACTTTGGATCATTATGCCACTGCCTGGTTCCGCCGGGCC |
| | CCAGGGAAGGAGCGTGAGGGGATCTCATATATTAGTAGTGGTGGTGGTAGCACGTACTAT |
| | GTAGACTCCGTGAAGGGCCGATTCACCATCTCCAGAGACAACGCCAAGAGGACGGTGTAT |
| | CTGCAAATGAACAGCCTGAAACCTGAGGACACAGCCGTTTATTTCTGTGCGTCCAAGAGA |
| | AGGTTCAATACTGGCTGGTCCGAGCCTTCAGATTATAACGTCTGGGGCCAGGGGACCCAG |
| | GTCACCGTCACCTCA |
| 45 | GCGGTACAACTGGTGGAGTCTGGGGGAGGCTTGGTGCAGCCTGGGGGGTCTCTGAGACTC |
| | TCCTGTGCAGCCTCCGGAAGTACAATCAGTATCAATGTCATGGGCTGGTACCGCCAGGCT |
| | CCAGGGAAGCAGCGCGACTTGGTCGCAGCTATTAGTAGTGTTGGTAGCACATACTATGCA |
| | GACTCCGTGAAGGGCCGATTCACCATCTCCAGAGACAACGCCAAGAACACGGTGTATCTG |
| | CAAATGAACAGCCTGAAACCTGAGGACACGGCCGTCTATTACTGTAATGCCAAGTCACTC |
| | ATAAGTGACTACTGGGGCCAGGGTACCCAGGTCACCGTCTCCTCA |
| 46 | CAGGTGCAGGTCGTGGAATCTGGGGGAGGATTGGTGCAGGCTGGGGGCTCTCTGAGACTC |
| | TCCTGTGCAGCCTCTGGACGCGCCTTCAGTAGCTATGCCATGGGCTGGTTCCGCCAGGCT |
| | CCAGGGAAGCAGCGCGAGTTGGTCGCAGTTATTACTAGTGGTGGTAGCACAAACTATGCA |
| | GACTCCGTGAAGGGCCGATTCACCATCTCCAGAGACAACGCCAAGAACACGGTGTATCTG |
| | CAAATGAACAGCCTGAAACCTGAGGACACGGCCGTTTATTACTGTGCAGCAGGGACCGGA |
| | TACGGGGGGGGGTTTTTAATTGGGTATGACTACTGGGGCCAGGGTACCCAGGTCACCGTC |
| | TCCTCA |
| 47 | CAGGTGCAGCTGGTGGAGTCTGGGGGAGGCTTGGTGCAGCCTGGGGGGTCCCTGAGACTC |
| | TCCTGTGCAGCCTCTGGAAGCCGCTTCATGACGAGTTCCATGGGCTGGTACCGCCAGACT |
| | CCAGGAAAACAGCGCGAGTGGGTCGCGACTGCTACTAGTAGTGATAGTACACTTTATGCA |
| | GACGCCGTGAAGGGCCGATTCACCATCTCCAGAGACAACGCCAAGAACACGGTGTATTTG |
| | CAAATGAACAGCCTGAAACCGCAGGACACGGCCGTCTATTACTGTTTCACAACACCACCC |
| | ACCTTCTGGGGCCACGGGACCCAGGTCACCGTCTCCTCA |
| 48 | GAGGTAGATCTTGTGGAGTCTGGGGGAGGCTTGGTGCAGCCTGGGGGGTCTCTGAGACTC |
| | TCCTGTGGAGCCTCTGGATTCACCTTCAGTCACTATGCCATGAGCTGGGTCCGCCAGGCT |
| | CCAGGAAAGGCACTCGAGTGGGTCTCAGATATTAGTAGTGGTGGTGGTATTACAAAGTAT |
| | GCAGACTCCGTGAAGGGCCGATTCACCATCTCCAGAGACGACGCCCAGAACACGCTGTAT |
| | CTGCAAATGAATAGCCTGAAACCTGAGGACACGGCCGTCTATTACTGTGCGAAAGCCAGT |
| | GCCATAGGATATCTCGCCTATTGGATGAGGAACCCCGTAGAGTATGACACCCGGGGACAG |
| | GGGACCCTGGTCACTGTCTCCTCA |
| 49 | CAGAAGCAGCTCGTGGAGTCTGGGGGAGGCTTGGTGCAGCCTGGGGGGTCTCTGAGACTC |
| | TCCTGTGCAGCCTCTGGAGAGACTTTGTCCAATTATAACATAGGCTGGTTCCGCCAGGCC |
| | CCAGGCAAGGAGCGTGAGGGGGTCTCATGCATTAGTCAGATTGGTTTCACCAACTATGCA |
| | GACTCCGTGAAGGGCCGATTCACCCTCTTTCGAGCGAGCGCCGAGAACACGGTGTATCTG |
| | CAAATGAACAGCCTGAAACCTGAGGACACAGCCGTTTATTACTGTGCAGTGACGGGCGGA |
| | AGATGGGCTAGCTGCAGTTTTCTGCCACTTCGCCAGGACTTTGAGTCCTGGGGCCAGGGG |
| | ACCCTGGTCACTGTCTCCTCA |
| 50 | GCGGTACAACTGGTGGAGTCTGGGGGAGGCATGGTGCAGCCGGGGGGGTCTCTGAGACTC |
| | TCCTGTGCAGCCTCTGGATTCACCTTCGGAGCCTATGACATGACCTGGGTCCGCCAGGCT |
| | CCAGGAAAGGGAGCCGAGTGGGTCTCAGCTATTAATAGTGGTGGTGACCGCACATACTAT |
| | ACAGACTCCGTGAAGGGCCGATTCACCATCTCCAGAGACAACGCCAAGAACACGCTGTAT |
| | CTGCAAATGAACAGCCTGAAACCTGCGGACACGGCCAAGTATTACTGTGGAACCAAAATC |
| | CCGCAGTTCCTCAGGGGCCAGGGGACCCAGGTCACCGTCTCCTCA |
| 51 | NYGMG |
| 52 | NYGMG |
| 53 | NYGMG |
| 54 | HYATA |
| 55 | AISSVGSTYYADSVKG |
| 56 | SYAMG |
| 57 | TSSMG |
| 58 | HYAMS |
| 59 | NYNIG |
| 60 | AYDMT |
| 61 | AISGGGVYTDFADAVKG |
| 62 | AISGGGVYRDFADSVKG |
| 63 | AISGGGVYTDFADSVKG |
| 64 | YISSGGGSTYYVDSVKG |
| 65 | AISSVGSTYYADSVKG |
| 66 | VITSGGSTNYADSVKG |
| 67 | TATSSDSTLYADAVKG |
| 68 | DISSGGGITKYADSVKG |
| 69 | CISQIGFTNYADSVKG |
| 70 | AINSGGDRTYYTDSVKG |
| 71 | ARTARVVSRRHEYDY |
| 72 | ARTPRVVNRRHEYDY |
| 73 | ARTARLTSRRHDYDY |
| 74 | KRRFNTGWSEPSDYNV |
| 75 | KSLISDY |
| 76 | GTGYGGGFLIGYDY |
| 77 | TPPTF |
| 78 | ASAIGYLAYWMRNPVEYDT |
| 79 | TGGRWASCSFLPLRQDFES |
| 80 | KIPQFL |
| 81 | SQVSQNCHNGSYEISVLMMGNSAFAEPLKNLEDAVNEGLEIVRGRLQNAGLNVTVNATFM |
| | YSDGLIHNSGDCRSSTCEGLDLLRKISNAQRMGCVLIGPSCTYSTFQMYLDTELSYPMIS |
| | AGSFGLSCDYKETLTRLMSPARKLMYFLVNFWKTNDLPFKTYSWSTSYVYKNGTETEDCF |
| | WYLNALEASVSYFSHELGFKVVLRQDKEFQDILMDHNRKSNVIIMCGGPEFLYKLKGDRA |
| | VAEDIVIILVDLFNDQYFEDNVTAPDYMKNVLVLTLSPGNSLLNSSFSRNLSPTKRDFAL |
| | AYLNGILLFGHMLKIFLENGENITTPKFAHAFRNLTFEGYDGPVTLDDWGDVDSTMVLLY |
| | TSVDTKKYKVLLTYDTHVNKTYPVDMSPTFTWKNSKLPNDITGRGPQ |
| 82 | EVOLVESGGGLVQPGGSLRLSCAASARPFSNYGMSWVRQAPGKGLEWVSAISGGGVYTYY |
| | ADSVKGRFTISRDNSKNTLYLQMNSLRAEDTAVYYCARARTARVVSRRHEYDYWGQGTQV |
| | TVSS |
| 83 | EVOLVESGGGLVQPGGSLRLSCAASARPFSNYGMGWFRQAPGKEREFVAAISGGGVYTYY |
| | ADSVKGRFTISRDNSKNTLYLQMNSLRAEDTAVYYCARARTARVVSRRHEYDYWGQGTQV |
| | TVSS |
| 84 | EVOLVESGGGLVQPGGSLRLSCTGSARPFSNYGMGWFRQAPGKEREFVAAISGGGVYTDF |
| | ADSVKGRFTISRDNSKNTLYLQMNSLRAEDTAVYYCARARTARVVSRRHEYDYWGQGTQV |
| | TVSS |
| 85 | EVOLVESGGGLVQPGGSLRLSCAASARPFSNYGMGWFRQAPGKEREFVAAISGGGVYTYY |
| | ADSVKGRFTISRDNSRNTLYLQMNSLRAEDTAVYYCARARTARVVSRRHEYDYWGQGTQV |
| | TVSS |
| 86 | EVOLVESGGGLVQPGGSLRLSCTGSARPFSNYGMGWFRQAPGNEREFVAAISGGGVYTDF |
| | ADSVKGRFTISRDNSKNTLYLQMNSLRAEDTAVYYCARARTARVVSRRHEYDYWGQGTQV |
| | TVSS |
| 87 | EVOLVESGGGLVQPGGSLRLSCAASGGTFVNYGMSWVRQAPGKGLEWVSAISGGGVYRYY |
| | ADSVKGRFTISRDNSKNTLYLQMNSLRAEDTAVYYCARARTPRVVNRRHEYDYWGQGTVQ |
| | TVSS |
| 88 | EVOLVESGGGLVQPGGSLRLSCAASGGTFVNYGMGWFRQAPGKEREFVAAISGGGVYRYY |
| | ADSVKGRFTISRDNSKNTLYLQMNSLRAEDTAVYYCARARTPRVVNRRHEYDYWGQGTQV |
| | TVSS |
| 89 | EVOLVESGGGLVQPGGSLRLSCTASGGTFVNYGMGWFRQAPGKEREFVAAISGGGVYRDF |
| | ADSVKGRFTISRDNSKNTLYLQMNSLRAEDTAVYYCARARTPRVVNRRHEYDYWGQGTQV |
| | TVSS |
| 90 | EVOLVESGGGLVQPGGSLRLSCAASGGTFVNYGMGWFRQAPGKEREFVAAISGGGVYRYY |
| | ADSVKGRFTISRDNSRNTLYLQMNSLRAEDTAVYYCARARTPRVVNRRHEYDYWGQGTQV |
| | TVSS |
| 91 | EVOLVESGGGLVQPGGSLRLSCAASGGIFSNYGMSWVRQAPGKGLEWVSAISGGGVYTYY |
| | ADSVKGRFTISRDNSKNTLYLQMNSLRAEDTAVYYCARARTARLTSRRHDYDYWGQGTQV |
| | TVSS |
| 92 | EVOLVESGGGLVQPGGSLRLSCAASGGIFSNYGMGWFRQAPGKEREFVAAISGGGVYTYY |
| | ADSVKGRFTISRDNSKNTLYLQMNSLRAEDTAVYYCARARTARLTSRRHDYDYWGQGTQV |
| | TVSS |
| 93 | EVOLVESGGGLVQPGGSLRLSCTASGGIFSNYGMGWFRQAPGKEREFVAAISGGGVYTDF |
| | ADSVKGRFTISRDNSKNTLYLQMNSLRAEDTAVYYCARARTARLTSRRHDYDYWGQGTQV |
| | TVSS |
| 94 | EVOLVESGGGLVQPGGSLRLSCAASGGIFSNYGMGWFRQAPGKEREFVAAISGGGVYTYY |
| | ADSVKGRFTISRDNSRNTLYLQMNSLRAEDTAVYYCARARTARLTSRRHDYDYWGQGTQV |
| | TVSS |
| 95 | QSQLVESGGGLVQPGGSLRLSCAASGSTLDHYAMSWVRQAPGKGLEWVSAISSGGGSTYY |
| | ADSVKGRFTISRDNSKNTLYLQMNSLRAEDTAVYYCASKRRENTGWSEPSDYNVWGQGTQ |
| | VTVTS |
| 96 | QSQLVESGGGLVQPGGSLRLSCAASGSTLDHYATAWFRRAPGKEREGISYISSGGGSTYY |
| | ADSVKGRFTISRDNSKNTLYLQMNSLRAEDTAVYYCASKRRFNTGWSEPSDYNVWGQGTQ |
| | VTVTS |
| 97 | QSQLVESGGGLVQPGGSLRLSCAASGSTLDHYATAWFRRAPGKEREGISYISSGGGSTYY |
| | VDSVKGRFTISRDNSKNTLYLQMNSLRAEDTAVYFCASKRRENTGWSEPSDYNVWGQGTQ |
| | VTVTS |
| 98 | QSQLVESGGGLVQPGGSLRLSCAASGSTLDHYATAWFRRAPGKEREGISYISSGGGSTYY |
| | ADSVAGRFTISRDNSRNTLYLQMNSLRAEDTAVYYCASKRRFNTGWSEPSDYNVWGQGTQ |
| | VTVTS |
| 99 | AVQLVESGGGLVQPGGSLRLSCAASGSTISINVMSWVRQAPGKGLEWVSVISSVGSTYYA |
| | DSVKGRFTISRDNSKNTLYLQMNSLRAEDTAVYYCNAKSLISDYWGQGTQVTVSS |
| 100 | AVQLVESGGGLVQPGGSLRLSCAASGSTISINVMGWYRQAPGKQRDLVAAISSVGSTYYA |
| | DSVKGRFTISRDNSKNTLYLQMNSLRAEDTAVYYCNAKSLISDYWGQGTQVTVSS |
| 101 | AVQLVESGGGLVQPGGSLRLSCAASGSTISINVMGWYRQAPGKQRDLVAAISSVGSTYYA |
| | DSVAGRFTISRDNSRNTLYLQMNSLRAEDTAVYYCNAKSLISDYWGQGTQVTVSS |
| 102 | EVOLVESGGGLVQPGGSLRLSCAASGRAFSSYAMSWVRQAPGKGLEWVSAITSGGSTYYA |
| | DSVKGRFTISRDNSKNTLYLQMNSLRAEDTAVYYCAAGTGYGGGFLIGYDYWGQGTQVTV |
| | SS |
| 103 | EVOLVESGGGLVQPGGSLRLSCAASGRAFSSYAMGWFRQAPGKQRELVAVITSGGSTYYA |
| | DSVKGRFTISRDNSKNTLYLQMNSLRAEDTAVYYCAAGTGYGGGFLIGYDYWGQGTQVTV |
| | SS |
| 104 | EVOLVESGGGLVQPGGSLRLSCAASGRAFSSYAMGWFRQAPGKQRELVAVITSGGSTNYA |
| | DSVKGRFTISRDNSKNTLYLQMNSLRAEDTAVYYCAAGTGYGGGFLIGYDYWGQGTQVTV |
| | SS |
| 105 | EVOLVESGGGLVQPGGSLRLSCAASGRAFSSYAMGWFRQAPGKQRELVAVITSGGSTYYA |
| | DSVKGRFTISRDNSRNTLYLQMNSLRAEDTAVYYCAAGTGYGGGFLIGYDYWGQGTQVTV |
| | SS |
| 106 | EVOLVESGGGLVQPGGSLRLSCAASGSRFMTSSMNWVRQAPGKGLEWVSTATSSDSTLYA |
| | DSVKGRFTISRDNAKNSLYLQMNSLRAEDTAVYYCFTTPPTFWGHGTQVTVSS |
| 107 | EVOLVESGGGLVQPGGSLRLSCAASGSRFMTSSMGWYRQTPGKQREWVATATSSDSTLYA |
| | DSVKGRFTISRDNAKNSLYLQMNSLRAEDTAVYYCFTTPPTFWGHGTQVTVSS |
| 108 | EVOLVESGGGLVQPGGSLRLSCAASGSRFMTSSMGWYRQTPGKQREWVATATSSDSTLYA |
| | DSVKGRFTISRDNARNSLYLQMNSLRAEDTAVYYCFTTPPTFWGHGTQVTVSS |
| 109 | EVOLVESGGGLVQPGGSLRLSCAASGSRFMTSSMGWYRQTPGNQREWVATATSSDSTLYA |
| | DSVKGRFTISRDNAKNSLYLQMNSLRAEDTAVYYCFTTPPTFWGHGTQVTVSS |
| 110 | EVOLVESGGGLVQPGGSLRLSCAASGSRFMTSSMGWYRQTPGNGREWVATATSSDSTLYA |
| | DSVKGRFTISRDNAKNSLYLQMNSLRAEDTAVYYCFTTPPTFWGHGTQVTVSS |
| 111 | EVOLVESGGGLVQPGGSLRLSCAASGFTFSHYAMSWVRQAPGKGLEWVSAISSGGGITYY |
| | ADSVKGRFTISRDNSKNTLYLQMNSLRAEDTAVYYCAKASAIGYLAYWMRNPVEYDTRGQ |
| | GTLVTVSS |
| 112 | EVQLVESGGGLVQPGGSLRLSCAASGFTFSHYAMSWVRQAPGKALEWVSDISSGGGITYY |
| | ADSVKGRFTISRDNSKNTLYLQMNSLRAEDTAVYYCAKASAIGYLAYWMRNPVEYDTRGQ |
| | GTLVTVSS |
| 113 | EVOLVESGGGLVQPGGSLRLSCGASGFTFSHYAMSWVRQAPGKALEWVSDISSGGGITKY |
| | ADSVKGRFTISRDNSKNTLYLQMNSLRAEDTAVYYCAKASAIGYLAYWMRNPVEYDTRGQ |
| | GTLVTVSS |
| 114 | QVQLVESGGGLVQPGGSLRLSCAASGFTFSHYAMSWVRQAPGKALEWVSDISSGGGITYY |
| | ADSVAGRFTISRDNSRNTLYLQMNSLRAEDTAVYYCAKASAIGYLAYWMRNPVEYDTRGQ |
| | GTLVTVSS |
| 115 | QVQLVESGGGLVKPGGSLRLSCAASGETLSNYNMSWIRQAPGKGLEWVSYISQIGFTNYA |
| | DSVKGRFTISRDNAKNSLYLQMNSLRAEDTAVYYCAVTGGRWASCSFLPLRQDFESWGQG |
| | TLVTVSS |
| 116 | QVQLVESGGGLVKPGGSLRLSCAASGETLSNYNIGWFRQAPGKEREGVSCISQIGFTNYA |
| | DSVKGRFTISRDNAKNSLYLQMNSLRAEDTAVYYCAVTGGRWASCSFLPLRQDFESWGQG |
| | TLVTVSS |
| 117 | QVQLVESGGGLVQPGGSLRLSCAASGETLSNYNIGWFRQAPGKEREGVSCISQIGFTNYA |
| | DSVKGRFTISRDNARNSLYLQMNSLRAEDTAVYYCAVTGGRWASCSFLPLRQDFESWGQG |
| | TLVTVSS |
| 118 | QPQVVESGGGLVQPGRSLTLSCAAYGTISKPYAMGWYRQAPEKQREWVATIFSDHSTNYA |
| | GAVKGRFTISRDNAKNTVYLQMNSLKPEDTAVYYCLPLGATFAGVAWGQGTQVTVSS |
| 119 | EVDLVESGGGLVQPGGSLRLSCAASQSIFSINVISWYRQAPGKQRELVAAIHSGGSTNYA |
| | DSVKGRFTISRDGTKNTVYLQMNSLKPEDTAVYYCHADTDFDRTFGSWGQGTQVTVSS |
| 120 | QVQLVESGGGLVQPGGSLRLSCAASGFTFGTYDMVWVRQAPGKGAEWVSGINSGGGRTYY |
| | ADSVKGRFTISRDNAKNTLYLQMNSLKSEDTAVYYCGTGDGRSRTYTGQGTQVTVSS |
| 121 | EVOLVESGGGLVQAGDSLRLSCAASGGTFSNYAMGWFRQAPGKDREFVSIVSQIGGVTRY |
| | ADSVKGRFTISRDNAKNMLYLQMNSLKPEDTAVYYCAAVRTNSRFYSDFEDSIVSWGQGT |
| | QVTVSS |
| 122 | EVOLVESGGGLVQPGGSLRLSCAASGSRFMTNAMGWWRQAPGKQREWVATATSNSTLYAE |
| | AVKGRFTISRDNAKNTVYLQMNSLKPQDTGVYYCFTTPPTAWGHGTQVTVSS |
| 123 | QVQLVESGGGVVQPGGSLRLSCAASGFTFGSYDMTWVRQAPGKGAEWVAAIDRGGTSTYY |
| | ADSVKGRFTISRDNAKNTLYLQMNSLKPEDTAVYYCGTREDGRATTGQGTQVTVSS |
| 124 | GTISKPYAMG |
| 125 | QSIFSINVIS |
| 126 | GFTFGTYDMV |
| 127 | GGTFSNYAMG |
| 128 | GSRFMTNAMG |
| 129 | GFTFGSYDMT |
| 130 | TIFSDHSTNYAGAVKG |
| 131 | AIHSGGSTNYADSVKG |
| 132 | GINSGGGRTYYADSVKG |
| 133 | IVSQIGGVTRYADSVKG |
| 134 | TATSNSTLYAEAVKG |
| 135 | AIDRGGTSTYYADSVKG |
| 136 | LGATFAGVA |
| 137 | DTDFDRTFGS |
| 138 | GDGRSRTY |
| 139 | VRTNSRFYSDFEDSIVS |
| 140 | TPPTA |
| 141 | REDGRAT |
| 142 | PYAMG |
| 143 | INVIS |
| 144 | TYDMV |
| 145 | NYAMG |
| 146 | TNAMG |
| 147 | SYDMT |
| 148 | TIFSDHSTNYAGAVKG |
| 149 | AIHSGGSTNYADSVKG |
| 150 | GINSGGGRTYYADSVKG |
| 151 | IVSQIGGVTRYADSVKG |
| 152 | TATSNSTLYAEAVKG |
| 153 | AIDRGGTSTYYADSVKG |
| 154 | LGATFAGVA |
| 155 | DTDFDRTFGS |
| 156 | GDGRSRTY |
| 157 | VRTNSRFYSDFEDSIVS |
| 158 | TPPTA |
| 159 | REDGRAT |
| 160 | CAGCCGCAGGTCGTGGAGTCTGGGGGAGGCTTGGTGCAGCCTGGGAGGTCTCTGACACTC |
| | TCCTGTGCAGCCTATGGAACCATCTCGAAGCCCTATGCCATGGGCTGGTACCGCCAGGCT |
| | CCAGAGAAGCAGCGCGAGTGGGTCGCAACTATTTTTAGTGACCATAGTACAAACTATGCA |
| | GGCGCCGTGAAGGGCCGATTCACCATTTCCAGAGACAATGCCAAGAACACGGTGTATCTG |
| | CAAATGAACAGCCTGAAACCTGAAGACACGGCCGTCTATTATTGTCTCCCATTGGGAGCG |
| | ACTTTTGCCGGTGTGGCCTGGGGCCAGGGGACCCAGGTCACCGTCTCCTCA |
| 161 | GAGGTAGATCTGGTGGAGTCTGGGGGAGGCTTGGTGCAGCCTGGGGGGTCTCTGAGACTC |
| | TCCTGTGCAGCCTCTCAAAGCATCTTCAGTATCAATGTCATATCCTGGTACCGCCAGGCT |
| | CCAGGGAAGCAGCGCGAGTTGGTCGCAGCTATTCATAGTGGTGGTAGCACAAACTATGCA |
| | GACTCCGTGAAGGGCCGATTCACCATCTCCAGAGACGGCACCAAGAATACGGTGTATCTG |
| | CAAATGAACAGCCTGAAACCTGAGGACACGGCCGTCTATTACTGTCATGCAGACACCGAC |
| | TTTGACCGGACCTTTGGTTCCTGGGGCCAGGGGACCCAGGTCACCGTCTCCTCG |
| 162 | CAGGTGCAGCTTGTGGAATCTGGGGGAGGCTTGGTGCAGCCTGGGGGTTCTCTGAGACTC |
| | TCCTGTGCAGCCTCTGGATTCACCTTCGGAACCTATGACATGGTCTGGGTCCGCCAGGCT |
| | CCAGGAAAGGGGGCCGAGTGGGTCTCAGGTATTAATAGTGGTGGTGGTCGGACATACTAT |
| | GCAGACTCCGTGAAGGGCCGCTTCACCATCTCCAGAGACAACGCCAAGAACACACTGTAT |
| | CTGCAAATGAACAGCCTGAAATCTGAGGACACGGCCGTGTATTATTGTGGAACAGGCGAC |
| | GGACGGTCCAGAACGTATACGGGCCAGGGGACCCAGGTCACCGTCTCCTCA |
| 163 | GAGGTACAACTCGTGGAGTCTGGGGGAGGATTGGTGCAGGCTGGAGACTCTCTGAGACTC |
| | TCCTGTGCAGCCTCTGGTGGCACCTTCAGTAACTACGCCATGGGCTGGTTCCGCCAGGCT |
| | CCAGGGAAGGACCGTGAGTTTGTCTCAATTGTTAGCCAGATTGGTGGTGTCACACGCTAT |
| | GCAGACTCCGTGAAGGGCCGATTCACCATCTCCAGAGACAACGCCAAGAATATGTTGTAT |
| | CTGCAAATGAACAGCCTGAAACCTGAGGACACAGCCGTCTATTACTGTGCGGCAGTAAGG |
| | ACTAATAGCAGGTTCTATAGCGACTTTGAAGATTCGATAGTTTCCTGGGGCCAGGGGACC |
| | CAGGTCACCGTCTCATCG |
| 164 | GAGGTGCAGCTTGTGGAATCTGGGGGAGGCTTGGTGCAGCCTGGGGGGTCTCTGAGACTC |
| | TCCTGTGCAGCCTCTGGAAGTCGCTTCATGACGAATGCCATGGGCTGGTGGCGCCAGGCT |
| | CCAGGAAAGCAGCGCGAGTGGGTCGCAACAGCTACTAGTAATAGTACGCTTTATGCAGAA |
| | GCCGTGAAGGGCCGATTCACCATCTCCAGAGACAACGCCAAGAACACGGTGTATTTGTTT |
| | CAAATGAACAGCCTGAAACCACAGGACACGGGCGTCTATTACTGCACAACACCACCCACC |
| | GCCTGGGGCCACGGGACCCAGGTCACCGTCTCCTCA |
| 165 | CAGGTGCAGCTTGTGGAATCTGGGGGAGGAGTGGTGCAGCCTGGGGGTTCTCTGAGACTC |
| | TCCTGTGCAGCCTCTGGATTCACCTTCGGAAGCTACGACATGACCTGGGTCCGCCAGGCT |
| | CCAGGAAAGGGGGCCGAGTGGGTCGCAGCTATAGATAGAGGTGGTACGAGCACATACTAT |
| | GCAGACTCCGTGAAGGGCCGATTCACCATCTCCAGAGACAACGCCAAGAACACGCTGTAT |
| | CTGCAAATGAACAGCCTGAAACCTGAGGACACGGCCGTGTATTACTGTGGAACTAGGGAG |
| | GACGGAAGAGCGACTACGGGCCAGGGGACCCAGGTCACCGTCTCCTCA |

1. An antibody specifically binding to Guanylate cyclase 2C (GCC) (anti-GCC antibody), comprising CDR1, CDR2 and CDR3 of a single domain antibody comprising an amino acid sequence selected from the group consisting of SEQ ID NOs: 1-10 and 118-123.

2. The antibody of claim 1, wherein the CDR1, CDR2 and CDR3 are according to IMGT numbering or Kabat numbering.

3.-4. (canceled)

5. An antibody specifically binding to Guanylate cyclase 2C (GCC) (anti-GCC antibody), wherein the anti-GCC antibody comprises:
(1) a CDR1 comprising the amino acid sequence of SEQ ID NO: 11, a CDR2 comprising the amino acid sequence of SEQ ID NO: 21, and a CDR3 comprising the amino acid sequence of SEQ ID NO: 31;
(2) a CDR1 comprising the amino acid sequence of SEQ ID NO: 12, a CDR2 comprising the amino acid sequence of SEQ ID NO: 22, and a CDR3 comprising the amino acid sequence of SEQ ID NO: 32;
(3) a CDR1 comprising the amino acid sequence of SEQ ID NO: 13, a CDR2 comprising the amino acid sequence of SEQ ID NO: 23, and a CDR3 comprising the amino acid sequence of SEQ ID NO: 33;
(4) a CDR1 comprising the amino acid sequence of SEQ ID NO: 14, a CDR2 comprising the amino acid sequence of SEQ ID NO: 24, and a CDR3 comprising the amino acid sequence of SEQ ID NO: 34;
(5) a CDR1 comprising the amino acid sequence of SEQ ID NO: 15, a CDR2 comprising the amino acid sequence of SEQ ID NO: 25, and a CDR3 comprising the amino acid sequence of SEQ ID NO: 35;
(6) a CDR1 comprising the amino acid sequence of SEQ ID NO: 16, a CDR2 comprising the amino acid sequence of SEQ ID NO: 26, and a CDR3 comprising the amino acid sequence of SEQ ID NO: 36;
(7) a CDR1 comprising the amino acid sequence of SEQ ID NO: 17, a CDR2 comprising the amino acid sequence of SEQ ID NO: 27, and a CDR3 comprising the amino acid sequence of SEQ ID NO: 37;
(8) a CDR1 comprising the amino acid sequence of SEQ ID NO: 18, a CDR2 comprising the amino acid sequence of SEQ ID NO: 28, and a CDR3 comprising the amino acid sequence of SEQ ID NO: 38;
(9) a CDR1 comprising the amino acid sequence of SEQ ID NO: 19, a CDR2 comprising the amino acid sequence of SEQ ID NO: 29, and a CDR3 comprising the amino acid sequence of SEQ ID NO: 39;
(10) a CDR1 comprising the amino acid sequence of SEQ ID NO: 20, a CDR2 comprising the amino acid sequence of SEQ ID NO: 30, and a CDR3 comprising the amino acid sequence of SEQ ID NO: 40;
(11) a CDR1 comprising the amino acid sequence of SEQ ID NO: 124, a CDR2 comprising the amino acid sequence of SEQ ID NO: 130, and a CDR3 comprising the amino acid sequence of SEQ ID NO: 136;
(12) a CDR1 comprising the amino acid sequence of SEQ ID NO: 125, a CDR2 comprising the amino acid sequence of SEQ ID NO: 131, and a CDR3 comprising the amino acid sequence of SEQ ID NO: 137;
(13) a CDR1 comprising the amino acid sequence of SEQ ID NO: 126, a CDR2 comprising the amino acid sequence of SEQ ID NO: 132, and a CDR3 comprising the amino acid sequence of SEQ ID NO: 138;
(14) a CDR1 comprising the amino acid sequence of SEQ ID NO: 127, a CDR2 comprising the amino acid sequence of SEQ ID NO: 133, and a CDR3 comprising the amino acid sequence of SEQ ID NO: 139;
(15) a CDR1 comprising the amino acid sequence of SEQ ID NO: 128, a CDR2 comprising the amino acid sequence of SEQ ID NO: 134, and a CDR3 comprising the amino acid sequence of SEQ ID NO: 140; or
(16) a CDR1 comprising the amino acid sequence of SEQ ID NO: 129, a CDR2 comprising the amino acid sequence of SEQ ID NO: 135, and a CDR3 comprising the amino acid sequence of SEQ ID NO: 141.

6. The anti-GCC antibody of claim 5, wherein the anti-GCC antibody comprises an amino acid sequence having at least 70% identity with an amino acid sequence selected from the group consisting of SEQ ID NOs: 1-10 and 82-123.

7. The anti-GCC antibody of claim 6, wherein the anti-GCC antibody comprises an amino acid sequence of any one selected from the group consisting of SEQ ID NOs: 1-10 and 82-123.

8. An anti-GCC antibody construct, comprising the anti-GCC antibody of claim 5.

9. The anti-GCC antibody construct of claim 8, further comprising an Fc fragment.

10. The anti-GCC antibody construct of claim 9, wherein the Fc fragment is selected from the group consisting of IgG1, IgG2, IgG3 and IgG4.

11. The anti-GCC antibody construct of claim 9, wherein the antibody construct is multi-specific.

12. The anti-GCC antibody construct of claim 11, wherein the anti-GCC antibody construct is bispecific.

13. An isolated nucleic acid encoding the anti-GCC antibody of claim 5.

14-15. (canceled)

16. A vector comprising the isolated nucleic acid of claim 13.

17. A host cell comprising the nucleic acid of claim 13 or a vector comprising thereof.

18. A conjugate comprising:
(1) the anti-GCC antibody of claim 5, and
(2) a drug.

19-23. (canceled)

24. A pharmaceutical composition, comprising:
(1) the anti-GCC antibody of claim 5, and
(2) a pharmaceutically acceptable excipient.

25. (canceled)

26. A method for treating a disorder associated with the undesired presence of GCC+ cells, comprising administrating an effective amount of the pharmaceutical composition of claim 24 to the subject.

27-28. (canceled)

29. A method of detecting GCC in a sample from a subject using the anti-GCC antibody of claim 5.
